SEQUENCE LISTING (8 sequences)

SEQ ID NO: 1 - DNA, 6087 bp, Artificial Sequence
  Synthetic polynucleotide
  misc_feature: Plasmid for AVXS-201 production
  misc_feature (1)..(106): mutated ITR
  misc_feature (151)..(699): 546 promotor fragment
  misc_feature (729)..(829): SV40 intron
  misc_feature (848)..(2344): hMECP2B
  misc_feature (2345)..(2393): synthetic pA
  misc_feature (2418)..(2558): ITR
  misc_feature (3309)..(4259): kanamycin resistance
  misc_feature (4325)..(4939): pMB1 ori
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaaataaaagatctttattttcattagatctgtgtgttggttttttgtgtggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacactatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgcc

SEQ ID NO: 2 - DNA, 549 bp, Artificial Sequence
  Synthetic polynucleotide
gtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaac

SEQ ID NO: 3 - DNA, 1497 bp, Artificial Sequence
  Synthetic polynucleotide
  misc_feature: MeCP2B cds
atggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctga

SEQ ID NO: 4 - DNA, 49 bp, Artificial Sequence
  Synthetic Polynucleotide
  misc_feature: Poly A sequence
aataaaagatctttattttcattagatctgtgtgttggttttttgtgtg

SEQ ID NO: 5 - DNA, 2558 bp, Artificial Sequence
  Synthetic Polynucleotide
  misc_feature: AVXS-201 genome
  misc_feature (1)..(106): mutated ITR
  misc_feature (151)..(699): 546 promoter fragment
  misc_feature (729)..(827): SV40 intron
  misc_feature (848)..(2344): hMECP2B cds
  misc_feature (2345)..(2393): synthetic pA
  misc_feature (2418)..(2558): ITR
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtaccacgcgtgaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaacgcgtcgaagggcgaattctgcagataactggtaagtttagtcttttttgtcttttatttcaggtcccggatccggtggtggtgcaaatcaaagaactgctcctcagtcgatgttgcctttacttctaggcctgtacggaagtgttactatggccgccgccgccgccgccgcgccgagcggaggaggaggaggaggcgaggaggagagactggaagaaaagtcagaagaccaggacctccagggcctcaaggacaaacccctcaagtttaaaaaggtgaagaaagataagaaagaagagaaagagggcaagcatgagcccgtgcagccatcagcccaccactctgctgagcccgcagaggcaggcaaagcagagacatcagaagggtcaggctccgccccggctgtgccggaagcttctgcctcccccaaacagcggcgctccatcatccgtgaccggggacccatgtatgatgaccccaccctgcctgaaggctggacacggaagcttaagcaaaggaaatctggccgctctgctgggaagtatgatgtgtatttgatcaatccccagggaaaagcctttcgctctaaagtggagttgattgcgtacttcgaaaaggtaggcgacacatccctggaccctaatgattttgacttcacggtaactgggagagggagcccctcccggcgagagcagaaaccacctaagaagcccaaatctcccaaagctccaggaactggcagaggccggggacgccccaaagggagcggcaccacgagacccaaggcggccacgtcagagggtgtgcaggtgaaaagggtcctggagaaaagtcctgggaagctccttgtcaagatgccttttcaaacttcgccagggggcaaggctgaggggggtggggccaccacatccacccaggtcatggtgatcaaacgccccggcaggaagcgaaaagctgaggccgaccctcaggccattcccaagaaacggggccgaaagccggggagtgtggtggcagccgctgccgccgaggccaaaaagaaagccgtgaaggagtcttctatccgatctgtgcaggagaccgtactccccatcaagaagcgcaagacccgggagacggtcagcatcgaggtcaaggaagtggtgaagcccctgctggtgtccaccctcggtgagaagagcgggaaaggactgaagacctgtaagagccctgggcggaaaagcaaggagagcagccccaaggggcgcagcagcagcgcctcctcaccccccaagaaggagcaccaccaccatcaccaccactcagagtccccaaaggcccccgtgccactgctcccacccctgcccccacctccacctgagcccgagagctccgaggaccccaccagcccccctgagccccaggacttgagcagcagcgtctgcaaagaggagaagatgcccagaggaggctcactggagagcgacggctgccccaaggagccagctaagactcagcccgcggttgccaccgccgccacggccgcagaaaagtacaaacaccgaggggagggagagcgcaaagacattgtttcatcctccatgccaaggccaaacagagaggagcctgtggacagccggacgcccgtgaccgagagagttagctgaaataaaagatctttattttcattagatctgtgtgttggttttttgtgtggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

SEQ ID NO: 6 - DNA, 106 bp, Artificial Sequence
  Synthetic polynucleotide
ctgcgcgctcgctcgctcactgaggccgcccgggcaaagcccgggcgtcgggcgacctttggtcgcccggcctcagtgagcgagcgagcgcgcagagagggagtgg

SEQ ID NO: 7 - DNA, 101 bp, Artificial Sequence
  Synthetic polynucleotide
ggtaagtttagtcttttttgtcttttatttcaggtcccggatccggtggtggtgcaaatcaaagaactgctcctcagtcgatgttgcctttacttctaggc

SEQ ID NO: 8 - DNA, 141 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtgg

(19) United States
(12) Patent Application Publication
(10) Pub. No.: US 2024/0189452 A1
(43) Pub. Date: Jun. 13, 2024

(54) Recombinant Adeno-Associated Virus Encoding Methyl-CPG Binding Protein 2 for Treating PITT Hopkins Syndrome VIA Intrathecal Delivery

(71) Applicant: RESEARCH INSTITUTE AT NATIONWIDE CHILDREN'S HOSPITAL, Columbus, OH (US)

(72) Inventors: Kathrin Christine Meyer, Columbus, OH (US); Cassandra Nicole Dennys-Rivers, Westerville, OH (US)

(21) Appl. No.: 18/286,626

(22) PCT Filed: Apr. 13, 2022
(86) PCT No.: PCT/US22/24644

§ 371 Date: Oct. 12, 2023

Related U.S. Application Data
(60) Provisional application No. 63/211,822, filed on Jun. 17, 2021.
(60) Provisional application No. 63/174,327, filed on Apr. 13, 2021.

(51) Int. Cl. A61K 48/00; A61K 38/17; A61P 25/00; C12N 15/86

(52) U.S. Cl. CPC A61K 48/005; A61K 38/1709; A61K 48/0075; A61P 25/00; C12N 15/86; C12N 2750/14143

(57) ABSTRACT

Methods and materials for treating Pitt Hopkins Syndrome comprising intrathecal delivery of recombinant Adeno-associated virus 9 (rAAV9) encoding Methyl-CpG binding protein 2 (MECP2) are provided.

This application claims priority to U.S. Provisional Patent Application No. 63/174,327 filed Apr. 13, 2021 and U.S. Provisional Patent Application No. 63/211,822 filed Jun. 17, 2021, which are incorporated by reference herein in their entirety.

### INCORPORATION BY REFERENCE OF MATERIAL SUBMITTED ELECTRONICALLY

Incorporated by reference in its entirety is a sequence listing in computer-readable form submitted concurrently herewith and identified as follows: ASCII text file named “56067_SeqListing.txt”, file size bytes 17, 282 created Apr. 12, 2022.

### FIELD OF THE INVENTION

The present invention relates to methods and materials for treating Pitt Hopkins Syndrome using recombinant adeno-associated virus 9 (rAAV9) encoding Methyl-CpG binding protein 2 (MECP2).

### BACKGROUND

Pitt Hopkins Syndrome (PTHS) is a neurological disorder caused by mutations in the TCF4 gene leading to haploinsufficiency affecting 1 in 34,000 to 41,000 individuals. Patients present with developmental delays, intellectual disabilities, microcephaly and seizures along with a broad spectrum of behavioral symptoms (Rosenfeld et al., Genet. Mut. 11:797-805, 2009). Unfortunately, due to the large size of the TCF4 gene and large number of splice variants, complete gene replacement therapy is currently not a viable option for treatment of PTHS. Therefore, there is a need to develop a novel therapeutic strategy for treating PTHS patients.

The MECP2 transcription factor modulates transcription of thousands of genes. MECP2 is a 52 kDa nuclear protein that is expressed in a variety of tissues but is enriched in neurons and has been studied most in the nervous system. There are two isoforms of MECP2 in humans known as MECP2A and B [Weaving et al., Journal of Medical Genetics, 42: 1-7 (2005)]. The two isoforms are derived from alternatively spliced mRNA transcripts and have different translation start sites. MECP2B includes exons 1, 3 and 4 and is the predominant isoform in the brain. MECP2 reversibly binds to methylated DNA and modulates gene expression [Guy et al., Annual Review of Cell and Developmental Biology, 27: 631-652 (2011)]. These functions map to the methyl binding domain (MBD) and transcriptional repressor domain (TRD), respectively [Nan & Bird, Brain & Development, 23, Suppl 1: S32-37 (2001)]. Originally thought of as a transcriptional repressor, MECP2 can both induce and suppress target gene expression [Chahrour et al., Science, 320: 1224-1229 (2008)]. MECP2 is hypothesized to support proper neuronal development and maintenance. In neurons, MECP2 facilitates translation of synaptic activity into gene expression through DNA binding and interaction with different binding partners [Ebert et al., Nature, 499: 341-345 (2013) and Lyst et al., Nature Neuroscience, 16: 898-902 (2013)]. In astrocytes, MECP2 deficiency is linked to apneic events in mice [Lioy et al., Nature, 475: 497-500 (2011)]. MECP2 deficiency can cause reduced brain size, increased neuronal packing density, reduced neuronal soma size and reduced dendritic complexity [Armstrong et al., Journal of Neuropathology and Experimental Neurology, 54: 195-201 (1995)]. Importantly, neuron death is not associated with MECP2 deficiency [Leonard et al., Nature Reviews, Neurology, 13: 37-51 (2017)]. MECP2 is also found outside the nervous system though levels vary across tissues.

Adeno-associated virus (AAV) is a replication-deficient parvovirus, the single-stranded DNA genome of which is about 4.7 kb in length including 145 nucleotide inverted terminal repeat (ITRs). The nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). Cis-acting sequences directing viral DNA replication (rep), encapsidation/packaging and host cell chromosome integration are contained within the ITRs. Three AAV promoters (named p5, p19. and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection. Multiple serotypes of AAV exist and offer varied tissue tropism. Known serotypes include, for example, AAV1, AAV2, AAV3, AAV4, AAV5, AAV6, AAV7, AAV8, AAV9, AAV10, AAV11, AAV12, AAV13 and AAVrh74. AAV9 is described in U.S. Pat. No. 7.198,951 and in Gao et al., J. Virol., 78: 6381-6388 (2004).

Interestingly, clinical features of PTHS overlap with Rett Syndrome, another autism spectrum disorder caused by mutations in the methyl CpG binding protein 2 (MECP2). The similarities in the disease phenotype can lead to misdiagnosis of PTHS patients. Indeed, a case study found reduced levels of MECP2 protein levels in blood samples of a PTHS patient (unpublished clinical data). Thus, there remains a need in the art for methods for treating both Rett Syndrome and PTHS and methods of disrupting the underlying pathways affected, which might lead to new therapeutic developments.

### SUMMARY

The present disclosure provides gene therapy methods and materials useful for treating Pitt Hopkins Syndrome (PTHS) in a patient in need thereof. In particular, the disclosure provides for a gene therapy vector expressing MeCP2 as a treatment for PTHS.

The disclosure provides for methods of treating PTHS comprising administering a recombinant adeno-associated virus (rAAV9) or a rAAV viral particle encoding Methyl-CpG binding protein 2 (MECP2) to a subject in need thereof. In some embodiments, the rAAV is administered by direct injection into the cerebrospinal fluid, intracerebroventricular delivery, intrathecal delivery or intravenous delivery. In some embodiments, the rAAV is administered to a patient in the Trendelenberg position. For example, the patient has a mutation in the TCF4 gene.

The disclosure provides for methods of increasing Methyl-CpG binding protein 2 (MECP2) levels in a subject suffering from PTHS comprising administering a recombinant adeno-associated virus (rAAV9) or a rAAV viral particle encoding MECP2 to the subject. In some embodiments, the rAAV is administered by direct injection into the cerebrospinal fluid, intracerebroventricular delivery, intrathecal delivery or intravenous delivery. In some embodiments, the rAAV is administered to a patient in the Trendelenberg position. For example, the patient has a mutation in the TCF4 gene.

The disclosure also provides for methods of delivering a polynucleotide sequence encoding the Methyl-CpG binding protein 2 (MECP2) to a subject suffering from PTHS comprising administering a recombinant adeno-associated virus (rAAV9) or a rAAV viral particle encoding MECP2 to the subject. In some embodiments, the rAAV is administered by direct injection into the cerebrospinal fluid, intracerebroventricular delivery, intrathecal delivery or intravenous delivery. In some embodiments, the rAAV is administered to a patient in the Trendelenberg position. For example, the patient has a mutation the TCF4 gene.

The disclosure also provides for methods and compositions for upregulating expression of the MECP2 protein in a subject suffering from PTHS, such upregulation may be induced by reactivation of the MECP2 gene.

In other embodiments, the patient is suffering from one or more of symptoms, wherein the symptom is intellectual disability including moderate intellectual disability or severe intellectual disability, developmental delay such as delayed development of mental and motor skills (psychomotor delay), breathing problems, recurrent seizures (epilepsy), and distinctive facial features, delayed or lack of speech or loss of speech, impaired communication skills, impaired socialization skills, hyperventilation, apnea, cyanosis, clubbing of fingers and/or toes, thin eyebrows, sunken eyes, strabismus, a prominent nose with a high nasal bridge, a pronounced double curve of the upper lip (cupid's bow), a wide mouth with full lips, widely spaced teeth, thick and/or cup-shaped ears, constipation, gastrointestinal problems, microcephaly, myopia, short stature, minor brain abnormalities, small hands and/or feet, single crease across the palm of the hands, pes planus, fleshy pads at the tips of the fingers/or toes, cryptorchidism, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems.

Exemplary involuntary hand movements include mechanical, repetitive hand movements, such as hand wringing, hand washing, or grasping.

Exemplary cardiac or heart problems include irregular heart rhythm. Such as abnormally long pauses between heartbeats, as measured by an electrocardiogram, or other types of arrhythmia.

The disclosure also provides for compositions for treating PTHS in a subject in need thereof wherein the composition comprises a rAAV or a rAAV viral particle encoding MECP2. In some embodiments, the composition is formulated for direct injection into the cerebrospinal fluid, intracerebroventricular delivery, intrathecal delivery or intravenous delivery. The disclosed compositions is administered to a patient in the Trendelenberg position. For example, the patient has a mutation in the TCF4 gene.

The disclosure provides for compositions for increasing Methyl-CpG binding protein 2 (MECP2) levels in a subject suffering from PTHS wherein the composition comprises a rAAV or a rAAV viral particle encoding MECP2. In some embodiments, the composition is formulated for direct injection into the cerebrospinal fluid, intracerebroventricular delivery, intrathecal delivery or intravenous delivery. The disclosed compositions is administered to a patient in the Trendelenberg position. For example, the patient has a mutation in the TCF4 gene.

The disclosure also provides for composition for delivering a polynucleotide sequence encoding the Methyl-CpG binding protein 2 (MECP2) to a subject suffering from PTHS wherein the composition comprises a rAAV or a rAAV viral particle encoding MECP2. In some embodiments, the composition is formulated for direct injection into the cerebrospinal fluid, intracerebroventricular delivery, intrathecal delivery or intravenous delivery. The disclosed compositions is administered to a patient in the Trendelenberg position. For example, the patient has a mutation in the TCF4 gene.

In other embodiments, the patient is suffering from one or more of symptoms, wherein the symptom is intellectual disability including moderate intellectual disability or severe intellectual disability, developmental delay such as delayed development of mental and motor skills (psychomotor delay), breathing problems, recurrent seizures (epilepsy), and distinctive facial features, delayed or lack of speech or loss of speech, impaired communication skills, impaired socialization skills, hyperventilation, apnea, cyanosis, clubbing of fingers and/or toes, thin eyebrows, sunken eyes, a prominent nose with a high nasal bridge, a pronounced double curve of the upper lip (cupid's bow), a wide mouth with full lips, widely spaced teeth, thick and/or cup-shaped ears, constipation, gastrointestinal problems, microcephaly, myopia, strabismus, short stature, minor brain abnormalities, small hands and/or feet, single crease across the palm of the hands, pes planus, fleshy pads at the tips of the fingers/or toes, cryptorchidism, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems.

In addition, the disclosure provide for use of a rAAV or a rAAV viral particle encoding MECP2 for the preparation of a medicament for the treatment of PTHS in a subject in need thereof. In some embodiments, the medicament is formulated for direct injection into the cerebrospinal fluid, intracerebroventricular delivery, intrathecal delivery or intravenous delivery. The disclosed medicament is administered to a patient in the Trendelenberg position. For example, the patient has a mutation in the TCF4 gene.

The disclosure provides for use of a rAAV or a rAAV viral particle encoding MECP2 for the preparation of a medicament for increasing Methyl-CpG binding protein 2 (MECP2) levels in a subject suffering from PTHS. In some embodiments, the medicament is formulated for direct injection into the cerebrospinal fluid, intracerebroventricular delivery, intrathecal delivery or intravenous delivery. The disclosed medicament is administered to a patient in the Trendelenberg position. For example, the patient has a mutation in the TCF4 gene.

The disclosure also provides for use of a rAAV or a rAAV viral particle encoding MECP2 for the preparation of a medicament for delivering a polynucleotide sequence encoding the Methyl-CpG binding protein 2 (MECP2) to a subject suffering from PTHS. In some embodiments, the medicament is formulated for direct injection into the cerebrospinal fluid, intracerebroventricular delivery, intrathecal delivery or intravenous delivery. The disclosed medicament administered to a patient in the Trendelenberg position. For example, the patient has a mutation in the TCF4 gene.

In other embodiments, the patient is suffering from one or more of symptoms, wherein the symptom is intellectual disability including moderate intellectual disability or severe intellectual disability, developmental delay such as delayed development of mental and motor skills (psychomotor delay), breathing problems, recurrent seizures (epilepsy), and distinctive facial features, delayed or lack of speech or loss of speech, impaired communication skills, impaired socialization skills, hyperventilation, apnea, cyanosis. clubbing of fingers and/or toes, thin eyebrows, sunken eyes, a prominent nose with a high nasal bridge, a pronounced double curve of the upper lip (cupid's bow), a wide mouth with full lips, widely spaced teeth, thick and/or cup-shaped ears, constipation, gastrointestinal problems, microcephaly, myopia, strabismus, short stature, minor brain abnormalities, small hands and/or feet, single crease across the palm of the hands, pes planus, fleshy pads at the tips of the fingers/or toes, cryptorchidism, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems.

In some embodiments, the rAAV administered in the disclosed methods, compositions or uses comprises a nucleotide sequence encoding MECP2, such as the nucleotide sequence of SEQ ID NO: 3. In addition, the rAAV comprises a nucleotide sequence that is at least 80%, 81%, 82%, 83%, 84%, 85%, 86%, 87%, 88%, 89%, 90%, 91%. 92%, 93%, 94%, 95%, 96%, 97%, 98% or 99% identical to the nucleotide sequence of SEQ ID NO: 3 and encodes a protein that retains MECP2 activity.

In addition, the disclosure provides for rAAV administered in the disclosed methods, compositions or uses further comprising the promoter sequence of SEQ ID NO: 2. For example, the rAAV comprises the promoter sequence of SEQ ID NO: 2 and the nucleotide sequence of SEQ ID NO: 3. The disclosure also provides rAAV further comprising an SV40 intron, a synthetic polyadenylation signal sequence and an inverted terminal repeat (ITR), such as a mutant ITR and a wild type ITR.

In an exemplary embodiment, the rAAV administered in the disclosed methods, compositions or uses comprises the nucleotide sequence of SEQ ID NO: 5 or nucleotides 151-2558 of SEQ ID NO: 1 or nucleotides 151 to 2393 or SEQ ID NO: 5. In addition, the rAAV comprises a nucleotide sequence that is at least 80%, 81%, 82%, 83%, 84%, 85%, 86%. 87%, 88%, 89%, 90%, 91%, 92%, 93%. 94%, 95%, 96%, 97%, 98% or 99% identical to the nucleotide sequence of SEQ ID NO: 5 or nucleotides 151-2558 of SEQ ID NO: 1 or nucleotides 151 to 2393 or SEQ ID NO: 5 and expresses a protein that retain MECP2 activity.

In any of the disclosed methods, compositions or uses, the rAAV is a AAV serotypes AAV1, AAV2, AAV3, AAV4, AAV5, AAV6, AAV7, AAV8, AAV9, AAV10, AAV11, AAV12, AAV13 or AAVrh74. In particular embodiments, the rAAV is serotype AAV9.

In any of the disclosed methods, compositions or uses, the patient is administered a composition comprising a disclosed rAAV and an agent that increases viscosity and/or density of the composition. For example, in some embodiments that agent is a contrast agent. The contrast agent may be 20 to 40% non-ionic, low-osmolar compound or contrast agent or about 25% to about 35% non-ionic, low-osmolar compound, such as iohexol, iobitridol, iomeprol, iopamidol, iopentol, iopromide, ioversol or ioxilan, or mixtures of two or more thereof. The disclosed composition may be formulated for any means of delivery, such as direct injection into the cerebrospinal fluid, intracerebroventricular delivery, intrathecal delivery or intravenous delivery.

In some embodiments, the patient is administered a composition comprising a disclosed rAAV the composition comprises an agent that increases the viscosity of the composition by about 0.05%, or by about 1% or by 1.5% or about 2% or by about 2.5% or by about 3% or by about 4% or by about 5% or by about 6% or by about 7% or by about 8% or by about 9% or by about 10%. In some embodiments, an agent increases the viscosity of the composition by about 1% to about 5%, or by about 2% to 12%, or by about 5% to about 10%, or by about 1% to about 20% or by about 10% to about 20%, or by about 10% to about 30%, or by about 20% to about 40%, or by about 20% to about 50%, or by about 10% to about 50%, or by about 1% to about 50%.

In some embodiments, the patient is administered a composition comprising a disclosed rAAV the composition comprises an agent that increases the density of the composition by about 0.05%, or by about 1% or by 1.5% or about 2% or by about 2.5% or by about 3% or by about 4% or by about 5% or by about 6% or by about 7% or by about 8% or by about 9% or by about 10%. In some embodiments, an agent increases the density of the composition by about 1% to about 5%, or by about 2% to 12%, or by about 5% to about 10%, or by about 1% to about 20%, or by about 10% to about 20%, or by about 10% to about 30%, or by about 20% to about 40% or by about 20% to about 50%, or by about 10% to about 50%, or by about 1% to about 50%.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). in some embodiments, the subject is a human. In some embodiments, the subject is a pediatric subject. In some embodiments, the subject is a pediatric subject, such as a subject ranging in age from 1 to 10 years. In some embodiments, the subject is 4 to 15 years of age. The subject, in on embodiment, is an adolescent subject, such as a subject ranging in age from 10 to 19 years. In other embodiments, the subject is an adult (18 years or older).

### BRIEF DESCRIPTION OF THE DRAWINGS

FIG. 1 provides a schematic of the rAAV9.P546.MECP2.

FIG. 2 demonstrates that PTHS induced Astrocytes (iAstrocytes) with TCF4 deletions have issues with differentiation. Representative images of iAstrocytes from healthy and TCF4 mutant cells following differentiation are provided.

FIG. 3 demonstrates that PTHS iAstrocytes with missense mutations have dysregulated TCF4 protein levels. or dysregulated protein isoforms, whereas deletion mutations have reduced TCF4 levels. Representative western blots of TCF4 levels within neuronal progenitor cells (A, NPCs) and iAstrocytes (B) show variable expression when normalized against control levels. TCF4 and GAPDH protein levels were quantified and normalized to healthy controls. Importantly, individual with gene deletions show reduction of TCF4 levels. Statistical analysis performed using one-way ANOVA against combined control data (N=3).

FIG. 4 demonstrates PTHS iAstrocytes produce abnormal neurite morphology and decreased motor neuron survival. Representative image of neurons (black) seeded on top of astrocytes (A). Neuronal quantification shows reduced survival, skeleton length and average neurite length (B).

FIG. 5 demonstrates PTHS NPCs have reduced MECP2 levels. Representative western blot of patient NPCs (N=3). MECP2 and GAPDH protein levels were quantified and normalized against healthy control lines. Statistical analysis was performed using on-way ANOVA (N=minimum of two experiments).

FIG. 6 demonstrates that TCF4 deletion mutation impairs iAstrocyte differentiation from Neuronal Progenitor Cells (NPCs) and transduction with AAV9.P546.MECP2 (10 and 100 MOI) two days prior to differentiation resulted in restored differentiation.

FIG. 7 demonstrates AAV9.P546.MECP2 was well tolerated in wild type (WT) mice. (A) Survival in WT mice treated with any vector dose is not significantly different from survival in untreated WT mice (p=0.1525) (Log-Rank/Mantel Cox test). (B) Severity score of untreated WT and vector treated WT mice shows that treatment overwhelmingly does not affect score.

FIG. 8 demonstrates that AAV9.P546.MECP2 treatment in wild type animals does not impair survival, behavior or ambulation. (A) At 60 days, vector treated WT mice do not have statistically different severity scores vs. untreated WT. p values: (untreated KO, p<0.0001. WT 1.50×10⁹, P>0.9999; WT 3.75×10⁹, p=0.9992: WT 7.50×10⁹, p>0.9999; WT 1.50×10¹⁰, p=0.9512; WT 3.00×10¹⁰, p=0.9876; WT 6.00×10¹⁰, p>0.9999. (B) At 90 days, vector treated WT mice do not have statistically different severity scores vs. WT, except 3.00×10¹⁰ vg. p values: (untreated KO, p<0.0001, 1.50×10⁹, p>0.9999; 3.75×10⁹, p=0.9911; 7.50×10⁹, p>0.8146; 1.50×10¹⁰, p=0.9983; 3.00×10¹⁰, p=0.0442; 6.00×10¹⁰, p>0.4566. (C-D) Open field assay for distance and velocity was performed at 49-63 days. (C) Vector treated WT mice do not have a significantly different open field velocity compared with untreated WT mice. p values vs. untreated WT (untreated KO, p<0.0001, 1.50×10⁹, p>0.9999; 3.75×10⁹, p>0.9999; 7.50×10⁹, p=0.9959; 1.50×10¹⁰, p=0.9991; 3.00×10¹⁰, p>9999; 6.00×10¹⁰, p>0.9999. (D) Vector treated WT mice do not have a significantly different open field distance compared with untreated WT mice. p values vs. untreated WT (untreated KO, p=0.0037, 1.5×10⁹, p>0.9999; 3.75×10⁹, p>0.9999; 7.5×10⁹, p=0.4199; 1.5×10¹⁰, p=0.9998; 3.0×10¹⁰, p=9976; 6.0×10¹⁰, p=0.7980. 60 and 90-day average severity scores were taken +/−2-4 days to account for slight time point variability in biweekly scoring intervals. WT untreated n=40, KO untreated n=43, WT-1.50×10⁹ n=11, WT-3.75×10⁹ n=32, WT-7.50×10⁹ n=16, WT-1.50×10¹⁰ n=36, WT-3.00×10¹⁰ n=20, WT-6.00×10¹⁰=18. Statistical significance was determined via ANOVA with Tukey's Test. Significance is in relation to untreated WT mice.

FIG. 9 demonstrates AAV9.P546.MECP2 produces dose dependent increases in MECP2 protein in wild type brains. A) Anti-MeCP2 western blots show a dose dependent elevation of total MeCP2 protein in various brain regions 3 weeks after P1 ICV injection. (Cb=cerebellum, Med=medulla, Hipp=hippocampus, Ctx=cortex, Mid=midbrain). TG3 indicates samples taken from a severe mouse model of MeCP2 Duplication Syndrome¹. B) Quantification of panel A. High, but not moderate, doses of AVXS-201 double MECP2 expression in select brain regions.

FIG. 10 demonstrates intrathecal infusion of AAV9.P546.MECP2 in non-human primates does not impair body weight growth. The three AVXS-201 treated animals are compared to the body weight for a control subject (circle).

FIG. 11 demonstrates intrathecal infusion of AAV9.P546.MECP2 in non-human primates does not impact hematology values through 18 months post injection. Values for the three AVXS-201 treated animals are compared to control subjects (circle).

FIG. 12 demonstrates intrathecal infusion of AAV9.P546.MECP2 in non-human primates does not impact serum chemistry through 12-18 months post injection. Liver and electrolyte values are similar between AAV9.P546.MECP2 treated and control treated subjects. Values for the three AAV9.P546.MECP2 treated animals are compared to control subjects (circle).

FIG. 13 demonstrates intrathecal infusion of AAV9.P546.MECP2 in non-human primates does not impact serum chemistry through 12-18 months post injection. Cardiac and renal values are similar between AAV9.P546.MECP2 treated and control treated subjects. Values for the three AAV9.P546.MECP2 treated animals are compared to control subjects (circle).

FIG. 14 demonstrates similar levels of MeCP2 expression throughout the brains of AAV9.P546.MECP2 treated and control non-human primates. Anti-MeCP2 immunohistochemistry revealed no gross structural abnormalities or obvious differences in MeCP2 expression. OC=Occipital Cortex, TC=Temporal Cortex, LSc=Lumbar spinal cord, Thal=Thalamus, Hipp=Hippocampus, Cb=Cerebellum.

FIG. 15 provides western blots of brain regions from control and AAV9.P546.MECP2 injected nonhuman primates show similar levels of MeCP2. Total MeCP2 levels and GAPDH loading controls are shown. Quantifications of panels A and B are shown below their respective blots. Dashed lines in the graphs indicate the average normalized values detected across controls. OC=Occipital Cortex, TC=Temporal Cortex, LSc=Lumbar spinal cord, Thal=Thalamus, Hipp=Hippocampus, Cb=Cerebellum. Values are shown as average±SEM.

FIG. 16 provides In situ hybridization showing vector derived transcript in all regions examined from brains of AAV9.P546.MECP2 treated nonhuman primates but not controls. The figure shows probes against GAPDH and vector derived MECP2 mRNA along with nuclear labeling (Dapi). OC=Occipital Cortex, TC=Temporal Cortex, LSc=Lumbar spinal cord, Hipp=Hippocampus, Cb=Cerebellum. Scale bars=20 μm.

FIG. 17 provides In situ hybridization shows vector derived transcript in all regions examined from brains of AVXS-201 treated nonhuman primates but not controls 18 months post injection. The figure shows probes against GAPDH and vector derived MECP2 mRNA along with nuclear labeling (Dapi). OC=Occipital Cortex, TC=Temporal Cortex, CA1 and CA3=Regions of the Hippocampus, CC=Corpus Callosum, Thal=Thalamus, Cau=Caudate, Put=Putamen, SColl=Superior Colliculus, Med=Medulla, Cb=Cerebellum, Cerv=cervical spinal cord, Thor=thoracic spinal cord, Lumb=lumbar spinal cord. Scale bars=20 μm.

FIG. 18 provides schematics and photos of the location of the ICV injection site in mice.

FIG. 19 provides microscopic views and photos of the location of the ICV injection site in mice.

FIG. 20 provides GFP protein expression in the brain after ICV injection of scAAV9.P546.GFP in mice.

FIG. 21 provides MeCP2 protein expression in the brain after ICV injection of scAAV9.P546.MeCP2 in wild type and TCF^{+/-} mice.

FIGS. 22 and 23 provide MeCP2 protein nuclear intensity in the Z-stack hippocampus and thalamus as recorded in different zones.

FIG. 24 provides graphs measuring the nuclear intensity in the anterior and posterior cortex, hippocampus, and thalamus.

FIG. 25 provides data from the marble burying test after ICV injection of scAAV9.P546.GFP in mice.

FIG. 26 provide data from the open field test after ICV injection of scAAV9.P546.GFP in mice.

FIG. 27 provides data from the elevated plus maze test after ICV injection of scAAV9.P546.GFP in mice.

### DETAILED DESCRIPTION

The present disclosure provides data using NPC and iAstrocytes obtained from PTHS patients which demonstrates that the patients had reduced expression of TCF4 and MECP2. Thus, the disclosure provides for methods of treating PTHS comprising administering an rAAV expressing MECP2.

rAAV are provided such as a self-complementary AAV9 (scAAV9) referred to herein as scAAV.P546.MECP2 or “AVXS-201.” Its gene cassette (nucleotides 151-2393 of the AVXS-201 genome set out in SEQ ID NO: 5) has, in sequence, a 546 bp promoter fragment (SEQ ID NO: 2) (nucleotides 74085586-74086323 of NC_000086.7 in the reverse orientation) from the mouse MECP2 gene, an SV40 intron, a human MECP2B cDNA (SEQ ID NO: 3) (CCDS Database #CCDS48193.1), and a synthetic polyadenylation signal sequence (SEQ ID NO: 4). The gene cassette is flanked by a mutant AAV2 inverted terminal repeat (ITR) and a wild type AAV2 inverted terminal repeat that together enable packaging of self-complementary AAV genomes. The genome lacks AAV rep and cap DNA, that is, there is no AAV rep or cap DNA between the ITRs of the genome.

TCF4 is implicated in maturation of oligodendrocytes as well as abnormal neuronal morphology (2-4) in Pitt Hopkins Syndrome (Li et al., Mol. Psych. 24: 1235-1246, 2019; Crux et al., PLOS One 13(6):1-9, 2018; Fu et al., J. Neurosci. 29: 11399-11408, 2009). However, the role of other cell types in the disorder is poorly understood. Using a direct conversion technology, human fibroblasts from patients with multiple neurological and neurodegenerative disorders were reprogrammed into neuronal progenitor cells (NPCs) and subsequently differentiated them into astrocytes (iAstrocytes) (Meyer et al., Proc. Natl. Acad. Sci. U.S.A. 111(2): 829-832, 2014). By co-culturing iAstrocytes with mouse neurons expressing GFP, a role for astrocytes in the disease pathology of a number of neurological disorders, including Rett Syndrome and Pitt Hopkins Syndrome has been demonstrated.

Interestingly, all PTHS patient cell lines showed downregulation of the transcription factor, methyl-CpG Binding Protein 2 (MeCP2). This is of particular importance as MeCP2 mutations lead to Rett syndrome which shares some clinical symptom overlap with PTHS patients. Furthermore, both transcription factors, MeCP2 and TCF4, have shared downstream pathways. Combined these observations suggest that increasing MeCP2 gene levels may be a strong alternative strategy for PTHS.

Together, these findings suggest that i) PTHS Astrocytes play a role in disease, ii) PTHS Astrocytes should be targeted therapeutically in addition to the neurons and iii) modulation of MECP2 levels, using a gene therapy construct is a potential therapeutic strategy for the treatment of PTHS. The disclosure provides for utilizing AAV9 p546.MECP2 construct to treat both astrocytes and/or neurons therapeutically.

In one aspect, the invention provides methods for the intrathecal administration (i.e., administration into the space under the arachnoid membrane of the brain or spinal cord) of a polynucleotide encoding MECP2 to a patient comprising administering a rAAV9 with a genome including the polynucleotide. In some embodiments, the rAAV9 genome is a self-complementary genome. In other embodiments, the rAAV9 genome is a single-stranded genome.

The methods deliver the polynucleotide encoding MECP2 to the brain and spinal cord of the patient (i.e., the central nervous system of the patient). Some target areas of the brain contemplated for delivery include, but are not limited to, the motor cortex and the brain stem. Some target cells of the central nervous system contemplated for delivery include, but are not limited to, nerve cells and glial cells. Examples of glial cells are microglial cells, oligodendrocytes and astrocytes.

“Treatment” comprises the step of administering via the intrathecal route an effective dose, or effective multiple doses, of a composition comprising a rAAV of the invention to a subject animal (including a human patient) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (either eliminates or reduces) at least one symptom associated with the disorder/disease state being treated, improves at least one symptom associated with the disorder/disease state being treated, that slows or prevents progression to a disorder/disease state, that diminishes the extent of disease, that results in remission (partial or total) of disease, and/or that prolongs survival.

In any of the methods, compositions and uses disclosed herein, the patient has a mutation in the gene encoding Transcription factor TCF4 (alias ITF2, SEF2 or E2-2) that results in impaired or reduced function of TCF4 protein. Missense, nonsense, frame-shift and splice-site mutations as well as translocations and large deletions encompassing TCF4 gene have been shown to cause Pitt-Hopkins syndrome (PTHS).

The TCF4 gene (MIM #610954) is located on chromosome 18q21.2, and it has 20 exons (the first and the last are noncoding) that span 360 kb. This transcription factor is a broadly expressed basic helix-loop-helix (bHLH) protein that functions as a homo- or heterodimer. The TCF4 exhibits transcription-regulatory activities that is highly expressed during early human development throughout the central nervous system, the sclerotome, peribronchial and kidney mesenchyme, and the genital bud, playing an important role in cellular proliferation, lineage commitment, and cellular differentiation.

Several alternatively spliced TCF4 variants have been described, allowing for the translation of at least 18 protein isoforms, with different N-terminal sequences. The following are exemplary mutations of the TCF4 gene known to cause PTHS: whole gene deletions, such as large rearrangements that are several megabases in size, partial gene deletions, such as deletions involving one or more of the exons from 7 to 20, balanced translocations, such as deletions disrupting the coding sequence of the gene, missense mutations, such as deletions involving the bHLH domain of TCF4, nonsense and frameshift mutations, such as mutations spread throughout the gene between exons 7 and 18, and slice site mutations, such as those affecting the donor and acceptor consensus splice sites and those that result in the shift off the reading frame. Exemplary genomic mutations include t(14;18)(q13.1;q21.2) and t(2;18)(q37;q21.2), which are de novo balanced translocations, respectively, with breakpoints falling within the second half of the gene. Additional exemplary mutations in the TCF4 gene are provided in Tables 1 and 2 below and are described in detail in Amiel et al. Am. J. Hum. Genet. 80(5):988-993, 2007, Pontual et al. Human Mut. 30:669-676, 2009, Goodspeed et al. J. Clin. Neurology 33(3): 233-244, 2018, and Zweier et al. J. Med. Genet. 45(11): 738-44, 2008 incorporated by reference herein in their entirety.

TABLE 1

| Type of mutation | DNA | Protein | Citation |
|---|---|---|---|
| frameshift | c.457_461del | | Goodspeed et al. |
| | c.520C > T | p.R174X | |
| | c.550-2A > G | | |
| deletion | c.624delc | | |
| | c.656-1G > C | | Zweier et al. |
| frameshift | c.680_682delinsT | W227LfsX29 | Goodspeed et al. |
| | c.692-694insT | p.G232fsX256 | Zweier et al. |
| Splice site | c.923-2A > G | | de Pontual et al. |
| frameshift | c.1031delA | | Goodspeed et al. |
| nonsense | c.1037C > G | | Goodspeed et al. |
| Splice site | c.1146 + 1G > A | | de Pontual et al. |
| | c.1153C > T | p.R385X | Zweier et al. |
| nonsense | c.1174 A > T | | Goodspeed et al. |
| frameshift | c.1239dupT | | Goodspeed et al. |
| frameshift | c.1414delG | | Goodspeed et al. |
| missense | c.1471A > G | p.D535G | de Pontual et al. |
| frameshift | c1472_1473insA | p.As-p462GlyfsX21 | de Pontual et al. |
| | c.1486 + 5 g > T | | |
| nonsense | c1498G > T | p.G500X | de Pontual et al. |
| frameshift | c.1521_1522insC | p.Ser508LeufsX5 | de Pontual et al. |
| missense | c.1650-2 A > G | | Goodspeed et al. |
| missense | c.1714G > A | p.R572G | de Pontual et al. |
| | c.1726C > T | p.R576W | Zweier et al., |
| | | | Amiel et al. |
| missense | c.1726C > G | p.R576G | de Pontual et al. |
| | c.1727G > A | p.R576Q | Amiel et al. |
| missense | c.1738C > T | p.Arg −> Try | Goodspeed et al. |
| missense | c.1739G > A | p.Arg −> Glu | Goodspeed et al. |
| missense | c.1823C > T | p.A610V | de Pontual et al. |
| frameshift | c.1933delG | | Goodspeed et al. |

TABLE 2

| | | | | | | | | |
|---|---|---|---|---|---|---|---|---|
| | | | Age at | Age at | | HV & | Stereo- | Sleep |
| Patient | Genotype | Genetic Test | Evaluation | Ambulation | Language | Apnea | typies | Disturbance |

FULL GENE DELETIONS

| | | | | | | | | |
|---|---|---|---|---|---|---|---|---|
| 3 | 4.2 Mb, ~16 | CMA 2016 | 1 y 10 m | − | − | − | + | − |
| | genes | (buccal) | | | | | | |
| 2 | 6.757 Mb, | CMA 2015 | 2 y 9 m | − | − | + | + | − |
| | ~20 genes | | | | | | | |
| 17 | 7.6 Mb, ~40 | CMA 2011 | 6 y 2 m | 6.5 y | − | − | + | + |
| | genes | | | | | | | |
| 8 | 25 Mb, ~100 | CMA 2012 | 28 y 7 m | − | − | − | UN | − |
| | genes | | | | | | | |

PARTIAL GENE DELETIONS

| | | | | | | | | |
|---|---|---|---|---|---|---|---|---|
| 21 | 148 kb- | CMA 2015 | 8 y 8 m | 1.5 y | Babble 24 m | − | + | − |
| | Exons 1 & | | | | | | | |
| | 2-, novel | | | | | | | |
| 4 | 20 kb- | CMA 2012 | 5 y 4 m | 5 y | Babble 6 m | − | + | − |
| | Exons 4 and 5 | | | | | | | |
| 14 | 188 kb- | CMA 2014 | 3 y 3 m | 2.5 y | Babble 20 m | − | + | + |
| | Exons 4 to 8 | | | | | | | |
| 19 | 100 kb- | CMA 2011 | 6 y 5 m | 3.5 y | − | + | + | − |
| | Exons 4 to 8, | | | | | | | |
| | de novo | | | | | | | |
| 15 | 138 kb- | CMA 2014 | 2 y 11 m | 2.5 y | Babble 11 m | - | + | + |
| | Exons 5 to 6, | | | | | | | |
| | similar | | | | | | | |
| | reported x1 | | | | | | | |
| 11 | 94 kb- | CMA 2010 | 10 y | 4 y | − | + | + | + |
| | Exons 5 to | | | | | | | |
| | 11 | | | | | | | |
| 7 | 3.8 kb- | CMA 2016 | 1 y 8 m | − | Babble 12 m | + | UN | + |
| | Exons 18, | | | | | | | |
| | 19, and part | | | | | | | |
| | of 20 | | | | | | | |

FRAMESHIFT MOLECULAR VARIANTS

| | | | | | | | | |
|---|---|---|---|---|---|---|---|---|
| 16 | c.680_ | Epilepsy Panel | 12 y 8 m | 9 y | Babbled 9 m | + | + | − |
| | 682delinsT, | 2012 | | | | | | |
| | Trp227Leufs | | | | | | | |
| | X29 in Exon | | | | | | | |
| | 10, novel | | | | | | | |
| 13 | c.457_ | WES 2013 | 18 y 11 m | 3.5 y | − | − | + | + |
| | 461del in Exon | | | | | | | |
| | 12, de novo, | | | | | | | |
| | novel | | | | | | | |
| 22 | c.1031delA | WES 2016 | 10 y 5 m | 9 y | − | + | + | − |
| | in Exon 13, | | | | | | | |
| | de novo, | | | | | | | |
| | nove | | | | | | | |
| 23 | c.1239dupT | Rett/AS Panel | | 3 y | − | + | + | + |
| | in Exon 15, | 2016 | | | | | | |
| | novel | | | | | | | |
| 1 | c.1414delG | Autism Panel 2015 | 3 y 4 m | − | Babble 24 m | UN | UN | − |
| | in Exon 16, | | | | | | | |
| | reported x1 | | | | | | | |
| 12 | c.1933delG | WES 2015 | 12 y 5 m | 1.3 y | Sentences 7 y | − | + | + |
| | in Exon 19, | | | | | | | |
| | de novo, | | | | | | | |
| | nove | | | | | | | |

MISSENSE MOLECULAR VARIANTS

| | | | | | | | | |
|---|---|---|---|---|---|---|---|---|
| 6 | c.1739G > A, | WES 2014 | 10 y 2 m | 10 y | − | + | + | + |
| | Arg −> Glu in | | | | | | | |
| | Exon 18, de | | | | | | | |
| | novo, | | | | | | | |
| | reported x2 | | | | | | | |
| 10 | c.17380 > T, | WES 2015 | 2 y 3 m | − | − | − | + | + |
| | Arg −> Try in | | | | | | | |
| | Exon 18, de | | | | | | | |
| | novo, | | | | | | | |
| | reported x6 | | | | | | | |
| 18 | c.1650-2 | Rett/AS Panel | 3 y | 3.5 y | − | + | + | + |
| | A > G in | 2015 | | | | | | |
| | intron 17 | | | | | | | |
| | leading to | | | | | | | |
| | splice site | | | | | | | |
| | variant, | | | | | | | |
| | novel, | | | | | | | |
| | c.1650-2 | | | | | | | |
| | A > C | | | | | | | |
| | reported x1 | | | | | | | |

NONSENSE-MOLECULAR VARIANTS

| | | | | | | | | |
|---|---|---|---|---|---|---|---|---|
| 9 | c.1037C > G | WES 2016 | 37 y 10 m | 3.5 y | − | + | + | UN |
| | in Exon 13, | | | | | | | |
| | de novo, | | | | | | | |
| | nove | | | | | | | |
| 20 | c.1174 A > T | TCF4 Sequence | 7 y 6 m | 5 y | − | − | − | − |
| | in Exon 15, | 2015 | | | | | | |
| | novel | | | | | | | |

DUPLICATIONS

| 5 | 201 kb | CMA 2016 | 2 y 4 m | − | Babble 12 m | − | − | + |
|---|---|---|---|---|---|---|---|---|
| | including | | | | | | | |
| | Exons 4 to 8, | | | | | | | |
| | mosaic | | | | | | | |
| | father, novel | | | | | | | |
| Total n(%) | | | | | | 10/22 (45) | 18/20 (90) | 12/22 (55) |
| | | | | | | Current | Marangi | Whalen |
| Patient | Genotype | Myopia | Strabismus | Constipation | Seizures | Medications | Score | Score |

FULL GENE DELETIONS

| | | | | | | | | |
|---|---|---|---|---|---|---|---|---|
| 3 | 4.2 Mb, ~16 | + | − | + | − | | 12 | 17 |
| | genes | | | | | | | |
| 2 | 6.757 Mb, | + | + | + | − | | 12 | 17 |
| | ~20 genes | | | | | | | |
| 17 | 7.6 Mb, ~40 | + | + | + | − | amantadine, | 12 | 18 |
| | genes | | | | | vitamin D | | |
| | | | | | | Dulcolax, iron, | | |
| | | | | | | Senna, | | |
| | | | | | | hydroxyzine | | |
| 8 | 25 Mb, ~100 | + | UN | + | + | h/o CBZ | 10 | 13 |
| | genes | | | | | | | |

PARTIAL GENE DELETIONS

| | | | | | | | | |
|---|---|---|---|---|---|---|---|---|
| 21 | 148 kb- | UN | UN | + | − | | 5 | 5 |
| | Exons 1 & 2- | | | | | | | |
| | novel | | | | | | | |
| 4 | 20 kb- Exons | + | + | − | + | OXC, LEV | 12 | 17 |
| | 4 and 5 | | | | | | | |
| 14 | 188 kb- | + | + | + | − | MiraLAX | 12 | 16 |
| | Exons 4 to 8 | | | | | | | |
| 19 | 100 kb- | + | + | + | − | amantadine, | 12 | 20 |
| | Exons 4 to 8, | | | | | MiraLAX, | | |
| | de novo | | | | | glycopyrolate, | | |
| | | | | | | lansoprazole | | |
| 15 | 138 kb- | UN | UN | + | + (GTCx1) | MiraLAX | 11 | 14 |
| | Exons 5 to 6, | | | | | | | |
| | similar | | | | | | | |
| | reported x1 | | | | | | | |
| 11 | 94 kb- Exons | + | + | + | + | amantadine, | 13 | 16 |
| | 5 to 11 | | | | | LTG, | | |
| | | | | | | Hydroxyzine, | | |
| | | | | | | OXC, Vayarin | | |
| 7 | 3.8 kb-Exons | + | + | − | − | MiraLAX, | 10 | 16 |
| | 18, 19, and | | | | | lansoprazole | | |
| | part of 20 | | | | | | | |

FRAMESHIFT MOLECULAR VARIANTS

| | | | | | | | | |
|---|---|---|---|---|---|---|---|---|
| 16 | c.680_ | UN | UN | − | − | acetazolamide, | 13 | 19 |
| | 682delinsT, | | | | | risperidone | | |
| | Trp227Leufsx | | | | | Vayarin | | |
| | 29 in Exon 10, | | | | | | | |
| | novel | | | | | | | |
| 13 | c.457_461del | + | − | + | − | clonidine, | 13 | 16 |
| | in Exon 12, de | | | | | risperidone | | |
| | novo, novel | | | | | | | |
| 22 | c.1031delA in | UN | UN | + | − | magnesium, | 11 | 18 |
| | Exon 13, de | | | | | amantadine | | |
| | novo, novel | | | | | | | |
| 23 | c.1239dupT in | + | + | + | − | risperidone, | 13 | 19 |
| | Exon 15, | | | | | melatonin, | | |
| | novel | | | | | amantadine | | |
| 1 | c.1414delG in | + | − | + | − | | 8 | 9 |
| | Exon 16, | | | | | | | |
| | reported x1 | | | | | | | |
| 12 | c.1933delG in | − | − | + | − | melatonin, | 9 | 11 |
| | Exon 19, de | | | | | MiraLAX | | |
| | novo, novel | | | | | | | |

MISSENSE MOLECULAR VARIANTS

| | | | | | | | | |
|---|---|---|---|---|---|---|---|---|
| 6 | c.1739G > A, | + | + | + | + | Seroquel | 14 | 19 |
| | Arg −> Glu in | | | | | | | |
| | Exon 18, de | | | | | | | |
| | novo, | | | | | | | |
| | reported x2 | | | | | | | |
| 10 | c.17380 > T, | + | + | + | − | | 12 | 16 |
| | Arg −> Try in | | | | | | | |
| | Exon 18, de | | | | | | | |
| | novo, | | | | | | | |
| | reported x6 | | | | | | | |
| 18 | c.1650-2 A > G | + | + | + | + | LEV, | 15 | 20 |
| | in intron 17 | | | | | glycopyrolate, | | |
| | leading to | | | | | lactulose, | | |
| | splice site | | | | | MiraLAX, | | |
| | variant, | | | | | risperidone, | | |
| | novel, c.1650- | | | | | melatonin, | | |
| | 2 A > C | | | | | esomeprazole, | | |
| | reported x1 | | | | | CBD oil | | |

NONSENSE-MOLECULAR VARIANTS

| | | | | | | | | |
|---|---|---|---|---|---|---|---|---|
| 9 | c.1037C > G in | UN | UN | + | + | amantadine, | 12 | 16 |
| | Exon 13, de | | | | | OXC, TPM, | | |
| | novo, novel | | | | | melatonin, | | |
| | | | | | | myoinositol, | | |
| | | | | | | CBD oil, | | |
| | | | | | | levothyroxine, | | |
| | | | | | | Mg, Kava, | | |
| | | | | | | Petadolex | | |
| 20 | c.1174 A > T in | − | + | + | + (IS) | pyridoxine, | 12 | 16 |
| | Exon 15, | | | | | clonidine, | | |
| | novel | | | | | amantadine | | |

DUPLICATIONS

| | | | | | | | | |
|---|---|---|---|---|---|---|---|---|
| 5 | 201 kb | UN | UN | − | − | melatonin | 9 | 14 |
| | including | | | | | | | |
| | Exons 4 to 8, | | | | | | | |
| | mosaic | | | | | | | |
| | father, novel | | | | | | | |

| | | | | |
|---|---|---|---|---|
| Total n(%) | 15/17 (88) | 12/16 (75) | 19/23 (83) | 8/23 (35) |

Cohort data from Goodspeed et al. J. Clin. Neurology 33(3): 233-244, 2018
Note is made of novel variants and inheritance pattern where available.
Patient 21 used TCF4 transcript variant 3 while the remainder of patients' genotypes were based on TCF4 transcript variant 1.
Abbreviations:
CMA-chromosomal microarray,
WES-whole exome sequencing,
CBZ-carbamazepine,
OXC-oxcarbazepine,
LEV-levetiracetam,
LTG-lamotrigine,
TPM-topiramate,
CBD-cannabidiol,
BP-base pair,
IS-infantile spasms,
UN-data unavailable at the time of chart review

In treatment of PTHS, the methods result in an effect in the subject including, but not limited to, improvement in muscle tone, improvement in walking and mobility, improvement in speech, reduction of breathing problems, reduction in apneas, reduction in seizures, reduction in anxiety, normalization of feeding behaviors, increased socialization, increase in IQ, normalization of sleep patterns and/or increased mobility.

Combination treatments are also contemplated by the invention. Combination as used herein includes both simultaneous treatment or sequential treatment. Combinations of methods of the invention with standard medical treatments for PTHS are specifically contemplated, as are combinations with novel therapies.

While delivery to an individual in need thereof after birth is contemplated, intrauteral delivery to a fetus is also contemplated.

In another aspect, the invention provides rAAV genomes. The rAAV genomes comprise one or more AAV ITRs flanking a polynucleotide encoding MECP2. The polynucleotide is operatively linked to transcriptional control DNAs, specifically promoter DNA and polyadenylation signal sequence DNA that are functional in target cells to form a “gene cassette.” The gene cassette may include promoters that allow expression specifically within neurons or specifically within glial cells. Examples include neuron specific enolase and glial fibrillary acidic protein promoters. Inducible promoters under the control of an ingested drug may also be used. Examples include, but are not limited to, systems such as the tetracycline (TET on/off) system [Urlinger et al., Proc. Natl. Acad. Sci. USA 97(14):7963-7968 (2000)] and the Ecdysone receptor regulatable system [Palli et al., Eur J. Biochem 270: 1308-1315 (2003]. The gene cassette may further include intron sequences to facilitate processing of an RNA transcript when the polynucleotide is expressed in mammalian cells.

The rAAV genomes of the invention lack AAV rep and cap DNA, that is, there is no AAV rep or cap DNA between the ITRs of the genomes. AAV DNA in the rAAV genomes (e.g., ITRs) may be from any AAV serotype for which a recombinant virus can be derived including, but not limited to, AAV serotypes AAV1, AAV2, AAV3, AAV4, AAV5. AAV6, AAV7, AAV8, AAV9, AAV10, AAV11, AAV12, AAV13 and AAVrh74. The nucleotide sequences of the genomes of the AAV serotypes are known in the art. For example, the AAV9 genome is provided in Gao et al., J. Virol., 78: 6381-6388 (2004).

In another aspect, the invention provides DNA plasmids comprising rAAV genomes of the invention. The DNA plasmids are transferred to cells permissible for infection with a helper virus of AAV (e.g., adenovirus, E1-deleted adenovirus or herpesvirus) for assembly of the rAAV genome into infectious viral particles with AAV9 capsid proteins. Techniques to produce rAAV particles, in which an AAV genome to be packaged, rep and cap genes, and helper virus functions are provided to a cell, are standard in the art. Production of rAAV requires that the following components are present within a single cell (denoted herein as a packaging cell): a rAAV genome, AAV rep and cap genes separate from (i.e., not in) the rAAV genome, and helper virus functions. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example. US 2005/0053922 and US 2009/0202490, the disclosures of which are incorporated by reference herein in their entirety.

### AAV Gene Therapy

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length including an inverted terminal repeat (ITRs). Exemplary ITR sequences may be 130 base pairs in length or 141 base pairs in length, such as the ITR sequence. There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45: 555-564 (1983) as corrected by Ruffing et al., J Gen Virol, 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5. p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non- dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Recombinant AAV genomes of the disclosure comprise nucleic acid molecule of the disclosure and one or more AAV ITRs flanking a nucleic acid molecule. AAV DNA in the rAAV genomes may be from any AAV serotype for which a recombinant virus can be derived including, but not limited to, AAV serotypes (e.g., AAV1, AAV2, AAV3, AAV4, AAV5, AAV6. AAV7, AAV8, AAV9, AAVRH10, AAVRH74, AAV11, AAV12, AAV13, or Anc80, AAV7m8 and their derivatives). Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). As noted in the Background section above, the nucleotide sequences of the genomes of various AAV serotypes are known in the art.

The provided recombinant AAV (i.e., infectious encapsidated rAAV particles) comprise a rAAV genome. The term “rAAV genome” refers to a polynucleotide sequence that is derived from a native AAV genome that has been modified. In some embodiments. the rAAV genome has been modified to remove the native cap and rep genes. In some embodiments, the rAAV genome comprises the endogenous 5′ and 3′ inverted terminal repeats (ITRs). In some embodiments, the rAAV genome comprises ITRs from an AAV serotype that is different from the AAV serotype from which the AAV genome was derived. In some embodiments, the rAAV genome comprises a transgene of interest flanked on the 5′ and 3′ ends by inverted terminal repeat (ITR). In some embodiments, the rAAV genome comprises a “gene cassette.” In exemplary embodiments, the genomes of both rAAV lack AAV rep and cap DNA, that is, there is no AAV rep or cap DNA between the ITRs of the genomes.

The rAAV genomes provided herein, in some embodiments, comprise one or more AAV ITRs flanking the transgene polynucleotide sequence. The transgene polynucleotide sequence is operatively linked to transcriptional control elements (including, but not limited to, promoters, enhancers and/or polyadenylation signal sequences) that are functional in target cells to form a gene cassette. Examples of promoters are the pIRF promoter, chicken β actin promoter (CBA), and the P546 promoter comprising the polynucleotide sequence set forth in SEQ ID NO: 2. Additional promoters are contemplated herein including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter.

Additionally provided herein are a P546 promoter sequence, and promoter sequences at least: 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence or P546 (SEQ ID NO: 2) sequence which exhibit transcription promoting activity.

Other examples of transcription control elements are tissue specific control elements, for example, promoters that allow expression specifically within neurons or specifically within astrocytes. Examples include neuron specific enolase and glial fibrillary acidic protein promoters. Inducible promoters are also contemplated. Non-limiting examples of inducible promoters include, but are not limited to a metallothionine promoter, a glucocorticoid promoter, a progesterone promoter, and a tetracycline-regulated promoter. The gene cassette may also include intron sequences to facilitate processing of a transgene RNA transcript when expressed in mammalian cells. One example of such an intron is the SV40 intron.

Conservative nucleotide substitutions in the rAAV9 genome including, but not limited to, in the gene cassette in the rAAV9 genome, are contemplated. For example, a MECP2 cDNA in a gene cassette may have 80%, 81%, 82%, 83%, 84%, 85%, 86%, 87%. 88%, 89%, 90%, 91%, 92%, 93%, 94%, 95%, 96%, 97%, 98% or 99% sequence identity to the MECP2 nucleotide sequence, such as the nucleotide sequence of SEQ ID NO: 3 that encodes a protein that retains MECP2 activity.

rAAV genomes provided herein comprises a polynucleotide (SEQ ID NO: 3) encoding MECP2 protein. In some embodiments, the rAAV genomes provided herein comprises a polynucleotide that encodes a polypeptide comprising an amino acid sequence that is at least: 90%, 91%, 92%, 93%, 94%, 95%, 96%, 97%, 98%, or 99% identical to the amino acid sequence encoded by the MECP2 cDNA.

rAAV genomes provided herein comprises a nucleotides 151-2393 of the nucleotide sequence set out as SEQ ID NO: 1 or nucleotides 151-2393 of the nucleotide sequence set out as SEQ ID NO: 5. In some embodiments, the rAAV genomes provided herein comprises a polynucleotide that at least: 90%, 91%, 92%, 93%, 94%, 95%, 96%, 97%. 98%, or 99% identical to the nucleotides 151-2393 of the nucleotide sequence set out as SEQ ID NO: 1 or nucleotides 151-2393 of the nucleotide sequence set out as SEQ ID NO: 5.

The terms “sequence identity”, “percent sequence identity”, or “percent identical” in the context of nucleic acid or amino acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. The percentage identity of the sequences can be determined by techniques known in the art. For example, homology can be determined by a direct comparison of the sequence information between two polypeptide molecules by aligning the sequence information and using readily available computer programs such as ALIGN, ClustalW2 and BLAST. In one embodiment, when BLAST is used as the alignment tool, the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR.

rAAV genomes provided herein, in some embodiments, a polynucleotide sequence that encodes an MECP2 protein and that hybridizes under stringent conditions to the polynucleotide sequence set forth in SEQ ID NO: 3 or the complement thereof.

DNA plasmids of the disclosure comprise rAAV genomes of the disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged. rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-9, AAV-1, AAV-2, AAV-3, AAV-4, AAV-5, AAV-6, AAV-7, AAVrh.74, AAV-8, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992. Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); McLaughlin et al., J. Virol., 62:1963 (1988); and Lebkowski et al., Mol. Cell. Biol., 7:349 (1988). Samulski et al., J. Virol., 63:3822-3828 (1989): U.S. Pat. No. 5,173,414; WO 95/13365 and corresponding U.S. Pat. No. 5,658.776 ; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The disclosure thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In another aspect, the invention contemplates compositions comprising a rAAV, such as a rAAV9, encoding a MECP2 polypeptide.

Compositions provided herein comprise rAAV and a pharmaceutically acceptable excipient or excipients. Acceptable excipients are nontoxic to recipients and are preferably inert at the dosages and concentrations employed, and include, but are not limited to, buffers such as phosphate [e.g., phosphate-buffered saline (PBS)], citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, copolymers such as poloxamer 188, pluronics (e.g., Pluronic F68) or polyethylene glycol (PEG). Compositions provided herein can comprise a pharmaceutically acceptable aqueous excipient containing a non-ionic, low-osmolar compound such as iobitridol, iohexol, iomeprol, iopamidol, iopentol, iopromide, ioversol, or ioxilan, where the aqueous excipient containing the non-ionic, low-osmolar compound can have one or more of the following characteristics: about 180 mgl/mL, an osmolality by vapor-pressure osmometry of about 322 mOsm/kg water, an osmolarity of about 273 mOsm/L, an absolute viscosity of about 2.3 cp at 20° C. and about 1.5 cp at 37° C., and a specific gravity of about 1.164 at 37° C.

Exemplary compositions comprise an agent to increase the viscosity and/or density of the composition. For example, the composition comprises a contrast agent to increase the viscosity and/or density of the composition. Exemplary compositions comprise about 20 to 40% non-ionic, low-osmolar compound or contrast agent or about 25% to about 35% non-ionic, low-osmolar compound. An exemplary composition comprises scAAV or rAAV viral particles formulated in 20 mM Tris (pH8.0), 1 mM MgCl₂, 200 mM NaCl, 0.001% poloxamer 188 and about 25% to about 35% non-ionic, low-osmolar compound. Another exemplary composition comprises scAAV formulated in and 1×PBS and 0.001% Pluronic F68.IG

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers and dosages of rAAV to be administered in methods of the invention will vary depending, for example, on the particular rAAV, the mode of administration, the treatment goal, the individual, the timing of administration, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). These dosages of rAAV may range from about 1×10⁹ vg or more, about 1×10¹⁰ vg or more, about 1×10¹¹ vg or more, about 1×10¹² vg or more, about 6×10¹² or more, about 1×10¹³ vg or more, about 1.3×10¹³ vg or more, about 1.4×10¹³ vg or more, about 2×10¹³ vg or more, about 3×10¹³ vg or more, about 6×10¹³ vg or more, about 1×10¹⁴ vg or more, about 3×10¹⁴ or more, about 6×10¹⁴ or more, about 1×10¹⁵ vg or more, about 3×10¹⁵ or more, about 6×10¹⁵ or more, about 1×10¹⁶ or more, about 3×10¹⁶ or more, or about 6×10¹⁶ or more. For a neonate, the dosages of rAAV may range from about 1×10⁹ vg or more, about 1×10¹⁰ vg or more, about 1×10¹¹ vg or more, about 1×10¹² vg or more, about 6×10¹² or more, about 1×10¹³ vg or more, about 1.3×10¹³ vg or more, about 1.4×10¹³ vg or more, about 2×10¹³ vg or more, about 3×10¹³ vg or more, about 6×10¹³ vg or more, about 1×10¹⁴ vg or more, about 3×10¹⁴ or more, about 6×10¹⁴ or more, about 1×10¹⁵ vg or more, about 3×10¹⁵ or more, about 6×10¹⁵ or more, about 1×10¹⁶ or more, about 3×10¹⁶ or more, or about 6×10¹⁶ or more.

Methods of transducing a target cell with rAAV, in vivo or in vitro, are contemplated by the disclosure. The in vivo methods comprise the step of administering an effective dose, or effective multiple doses, of a composition comprising a rAAV of the disclosure to an animal (including a human being) in need thereof. If the dose is administered prior to development of a disorder/disease, the administration is prophylactic. If the dose is administered after the development of a disorder/disease, the administration is therapeutic. In embodiments of the disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. Example of a disease contemplated for prevention or treatment with methods of the disclosure is PTHS.

Transduction of cells using rAAV of the invention results in sustained expression of the MECP2 polypeptide encoded by the rAAV. In some embodiments, the target expression level is contemplated to be about 10% to about 25% of the normal (or wild type) physiological expression level in a subject who does not have PTHS, or about 25% to about 50% of the normal (or wild type) physiological expression level in a subject who does not have PTHS, or about 50% to about 75% of the normal (or wild type) physiological expression level in a subject who does not have PTHS or about 75% to about 125% of the normal (or wild type) physiological expression level in a subject who does not have PTHS. The target expression level may be about 10%, about 15%, about 20%, about 25%, about 30%, about 35%, about 40%, about 45%, about 50%, about 55%, about 60%, about 65%, about 70% about 75%, about 80%, about 85%, about 90%, about 95%, about 100%, about 105%, about 110%, about 115%, about 120% or about 125% of the normal expression level.

The term “transduction” is used to refer to the administration/delivery of the coding region of the MECP2 to a recipient cell either in vivo or in vitro, via a replication- deficient rAAV of the disclosure resulting in expression of MECP2 in the recipient cell.

In some embodiments of treatment methods of the invention, an agent that increases viscosity and/or density of the composition is administered to the patient. For example, a non-ionic, low-osmolar contrast agent is also administered to the patient. Such contrast agents include, but are not limited to, iobitridol, iohexol, iomeprol, iopamidol, iopentol, iopromide, ioversol, ioxilan, and mixtures of two or more of the contrast agents. In some embodiments, the treatment methods thus further comprise administration of iohexol to the patient. The non-ionic, low-osmolar contrast agent is contemplated to increase transduction of target cells in the central nervous system of the patient. It is contemplated that the transduction of cells is increased when a rAAV of the disclosure is used in combination with a contrast agent as described herein relative to the transduction of cells when a rAAV of the disclosure is used alone. In various embodiments, the transduction of cells is increased by at least about 1%, or at least about 5%, at least about 10%, at least about 20%, at least about 30%, at least about 40%, at least about 50%, at least about 60%, at least about 70%, at least about 80%, at least about 90%, at least about 100%, at least about 120%, at least about 150%, at least about 180%, at least about 200%, at least about 250%, at least about 300%, at least about 350%, at least about 400%, at least about 450%, at least about 500% or more when a vector of the disclosure is used in combination with a contrast agent as described herein, relative to the transduction of a vector of the disclosure when not used in combination with a contrast agent. In further embodiments, the transduction of cells is increased by about 10% to about 50%, or by about 10% to about 100%, or by about 5% to about 10%, or by about 5% to about 50%, or by about 1% to about 500%, or by about 10% to about 200%, or by about 10% to about 300%, or by about 10% to about 400%, or by about 100% to about 500%, or by about 150% to about 300%, or by about 200% to about 500% when a vector of the disclosure is used in combination with a contrast agent as described herein, relative to the transduction of a vector of the disclosure when not used in combination with a contrast agent.

In some embodiments, it is contemplated that the transduction of cells is increased when the patient is put in the Trendelenberg position (head down position). In some embodiments, for example, the patients is tilted in the head down position at about 1 degree to about 30 degrees, about 15 to about 30 degrees, about 30 to about 60 degrees. about 60 to about 90 degrees, or about 90 up to about 180 degrees) during or after intrathecal vector infusion. In various embodiments, the transduction of cells is increased by at least about 1%, or at least about 5%, at least about 10%, at least about 20%, at least about 30%, at least about 40%, at least about 50%, at least about 60%, at least about 70%, at least about 80%, at least about 90%, at least about 100%, at least about 120%, at least about 150%, at least about 180%, at least about 200%, at least about 250%, at least about 300%, at least about 350%, at least about 400%, at least about 450%, at least about 500% or more when a the Trendelenberg position is used as described herein, relative to when the Trendelenberg position is not used.

In further embodiments, the transduction of cells is increased by about 10% to about 50%, or by about 10% to about 100%, or by about 5% to about 10%, or by about 5% to about 50%, or by about 1% to about 500%, or by about 10% to about 200%, or by about 10% to about 300%, or by about 10% to about 400%, or by about 100% to about 500%, or by about 150% to about 300%, or by about 200% to about 500% when a vector of the disclosure is used in combination with a contrast agent and the Trendelenberg position as described herein, relative to the transduction of a vector of the disclosure when not used in combination with a contrast agent and Trendelenberg position.

The disclosure also provides treatment method embodiments wherein the intrathecal administration of a vector of the disclosure and a contrast agent to the central nervous system of a patient in need thereof who is put in the Trendelenberg position results in a further increase in survival of the patient relative to survival of the patient when a vector of the disclosure is administered in the absence of the contrast agent and the Trendelenberg position. In various embodiments, administration of a vector of the disclosure and a contrast agent to the central nervous system of a patient in need thereof put in the Trendelberg position results in an increase of survival of the patient of at least about 1%, at least about 5%, at least about 10%, at least about 20%, at least about 30%, at least about 40%, at least about 50%, at least about 60%, at least about 70%, at least about 80%, at least about 90%, at least about 100%, at least about 150%, at least about 200% or more relative to survival of the patient when a vector of the disclosure is administered in the absence of the contrast agent and the Trendelenberg position.

Combination therapies are also contemplated by the disclosure. Combination as used herein includes both simultaneous treatment and sequential treatments. Combinations of methods of the disclosure with standard medical treatments are specifically contemplated, as are combinations with novel therapies. In some embodiments, the combination therapy comprises administering an immunosuppressing agent in combination with the gene therapy disclosed herein.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the disclosure may be chosen and/or matched by those skilled in the art taking into account the disease state being treated and the target cells/tissue(s) that are to express the MECP2 protein.

The disclosure provides for local administration and systemic administration of an effective dose of rAAV and compositions of the disclosure. For example, systemic administration is administration into the circulatory system so that the entire body is affected. Systemic administration includes enteral administration such as absorption through the gastrointestinal tract and parenteral administration through injection, infusion or implantation.

### Immunosuppressing Agents

The immunosuppressing agent may be administered before or after the onset of an immune response to the rAAV in the subject after administration of the gene therapy. In addition, the immunosuppressing agent may be administered simultaneously with the gene therapy or the protein replacement therapy. The immune response in a subject includes an adverse immune response or an inflammatory response following or caused by the administration of rAAV to the subject. The immune response may be the production of antibodies in the subject in response to the administered rAAV.

Exemplary immunosuppressing agents include glucocorticosteroids, janus kinase inhibitors, calcineurin inhibitors, mTOR inhibitors, cyctostatic agents such as purine analogs, methotrexate and cyclophosphamide, inosine monophosphate dehydrogenase (IMDH) inhibitors, biologics such as monoclonal antibodies or fusion proteins.

The immunosuppressing agent may be an anti-inflammatory steroid, which is a steroid that decreases inflammation and suppresses or modulates the immune system of the subject. Exemplary anti-inflammatory steroid are glucocorticoids such as prednisolone, betamethasone, dexamethasone, hydrocortisone, methylprednisolone, deflazacort, budesonide or prednisone.

Janus kinase inhibitors are inhibitors of the JAK/STAT signaling pathway by targeting one or more of the Janus kinase family of enzymes. Exemplary janus kinase inhibitors include tofacitinib, baricitinib, upadacitinib, peficitinib, and oclacitinib.

Calcineurin inhibitors bind to cyclophilin and inhibits the activity of calcineurin Exemplary calcineurine inhibitors includes cyclosporine, tacrolimus and picecrolimus.

mTOR inhibitors reduce or inhibit the serine/threonine-specific protein kinase mTOR. Exemplary mTOR inhibitors include sirolimus, everolimus, and temsirolimus.

The immunosuppressing agents include immune suppressing macrolides. The term “immune suppressing macrolides” refer to macrolide agents that suppresses or modulates the immune system of the subject. A macrolide is a class of agents that comprise a large macrocyclic lactone ring to which one or more deoxy sugars, such as cladinose or desoamine, are attached. The lactone rings are usually 14-, 15-, or 16-membered. Macrolides belong to the polyketide class of agents and may be natural products. Examples of immunosuppressing macrolides include tacrolimus, pimecrolimus, and sirolimus.

Purine analogs block nucleotide synthesis and include IMDH inhibitors. Exemplary purine analogs include azathioprine, mycophenolate and lefunomide.

Exemplary immunosuppressing biologics include abatacept, adalimumab, anakinra, certolizumab, etanercept, golimumab, infliximab, ixekizumab, natalizumab, rituximab, secukinumab, tocilizumab, ustekinenumab, vedolizumab, basiliximab, belatacep, and daclizumab.

In particular, the immunosuppressing agent is an anti-CD20 antibody. The term anti-CD20 specific antibody refers to an antibody that specifically binds to or inhibits or reduces the expression or activity of CD20. Exemplary anti-CD20 antibodies include rituximab, ocrelizumab or ofatumumab.

Additional examples of immuosuppressing antibodies include anti-CD25 antibodies (or anti-IL2 antibodies or anti-TAC antibodies) such as basiliximab and daclizumab, and anti-CD3 antibodies such as muromonab-CD3, otelixizumab, teplizumab and visilizumab, anti-CD52 antibodies such as alemtuzumab.

The following EXAMPLES are provided by way of illustration and not limitation. Described numerical ranges are inclusive of each integer value within each range and inclusive of the lowest and highest stated integer.

### EXAMPLES

### Example 1

TCF4 Protein Levels are Variable within Individuals with Missense Mutations.

Direct conversion of patient fibroblasts to neuronal progenitor cells (NPCs) allows for the study of disease mechanism in specific cell types of interest. This in vitro cell model can be used to distinguish patient responders based on the presence of specific disease markers of cellular stress. If disease markers are present, this information can then be used to choose potential therapeutics from a selection of therapeutic molecules, such as small molecules or biologics to determine their effect on the PTHS phenotype.

Fibroblasts from six PTHS patients containing either heterozygous missense or deletion mutations in TCF4 were obtained and are summarized in Table 3 below. The fibroblasts were converted to induced neuronal progenitor cells (iNPCs) using retroviruses, SOX2, KLF4, cMyc, and Oct3/4, and chemically defined media as previously described (Meyer et al., PNAS 829-832 (2014)). Subsequently, the NPCs were differentiation into astrocytes (iAstrocytes). Neuronal progenitor cells were cultured on fibronectin coated dishes in NPC media (DMEM/F12 media containing 1% N2 supplement (Life Technologies), 1% B27, 1% Anti-anti (antibiotic-antimycotic) 20 ng/ml fibroblast growth factor-2) until onfluent. iAstrocytes were differentiated by seeding a small quantity of NPCs on another fibronectin coated dish in astrocyte inducing media (DMEM media containing 0.2% N2). These induced astrocytes are referred to as iastrocytes or iAST herein. Neurons were converted from NPCs by transduction with retro-Ngn2.

TABLE 3

| Cell | | |
|---|---|---|
| Line | Sex | Mutation |
| TCF4-1 | female | c.1486 + 5 g > T |
| TCF4-2 | male | c.520C > T(p.Arg174X) |
| TCF4-3 | male | Heterozygous gene deletion |
| | | (del(18)(q21.2q21.32) |
| TCF4-4 | female | c.1726C > T (p.Arg576X) |
| TCF4-5 | male | c.624delc |
| TCF4-6 | male | c.550 − 2A > G |

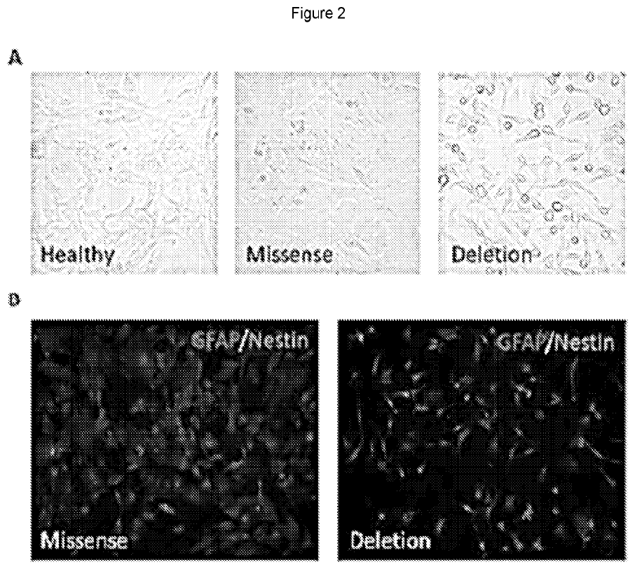

Five days post differentiation, induced astrocytes were seeded either into a 96 well (10,000 cells/well), 384 well (2,500 cells/well), a 24 well seahorse plate (20,000 cells/well) or a 96 well seahorse plate (10,000 cells/well). A representative image of iAstrocytes from healthy and TCF4 mutants following differentiation are provided in FIG. 2.

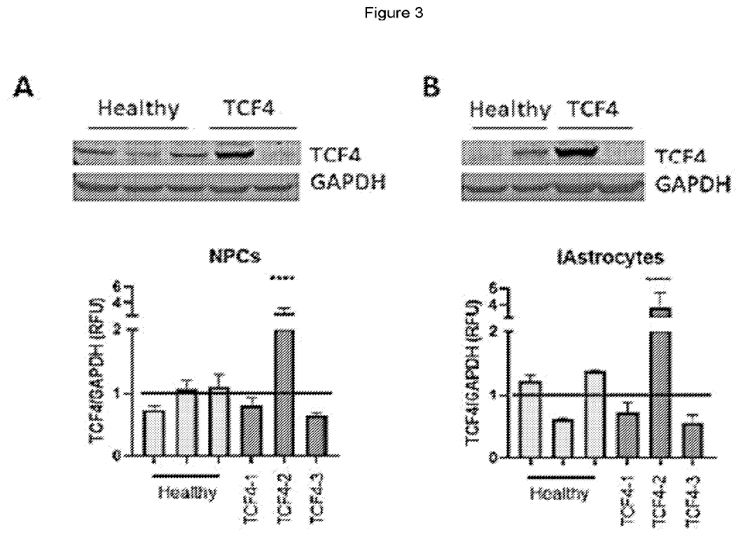

Initial studies on three of these patient lines investigated the levels of TCF4 protein in patient neuronal progenitor cells and iAstrocytes. Western blot of TCF4 (isoforms B, D, E, F, M, N, O, Q) discovered differential levels in PTHS iAstrocytes and NPCs compared to healthy controls (FIGS. 3A and B). Importantly, patients with heterozygous genetic deletions had 50% reduction in TCF4 levels whereas missense mutations either lead to no change in protein levels or significant upregulation, potentially suggesting toxic overexpression (FIG. 3B).

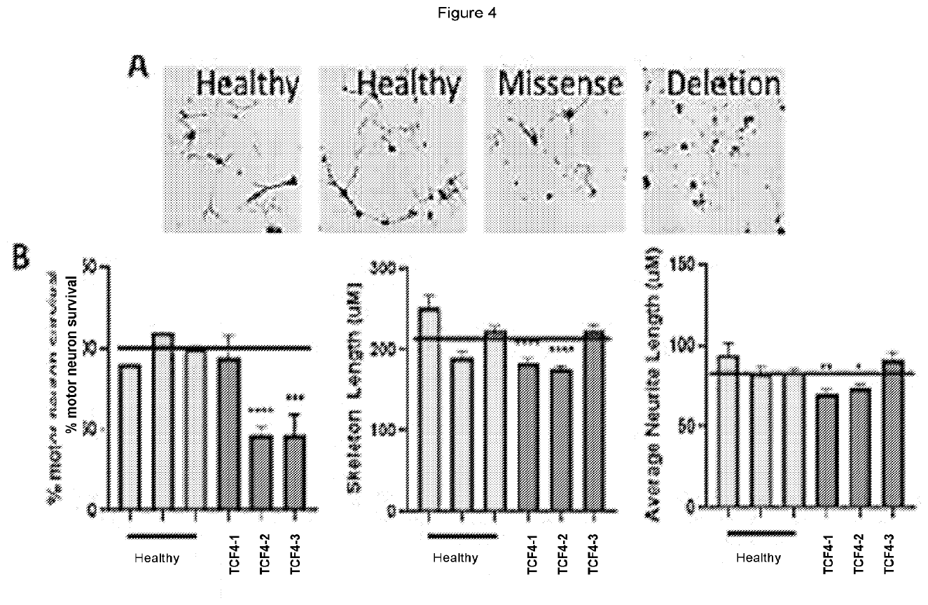

In addition, GFP+ neurons co-cultured with iAstrocytes from TCF4 patients show reduced neuronal survival (FIGS. 4A and B). PTHS iAstrocytes caused changes in neuronal morphology (FIG. 4B). Thus, this direct conversion technology and co-culture assay can be utilized to identify new disease mechanisms as well as evaluate potential therapeutic strategies (including but not limited to gene therapy) to treat patients with PTHS.

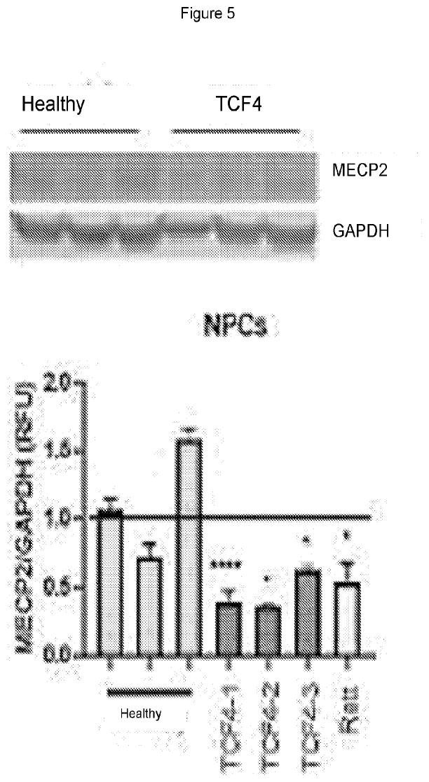

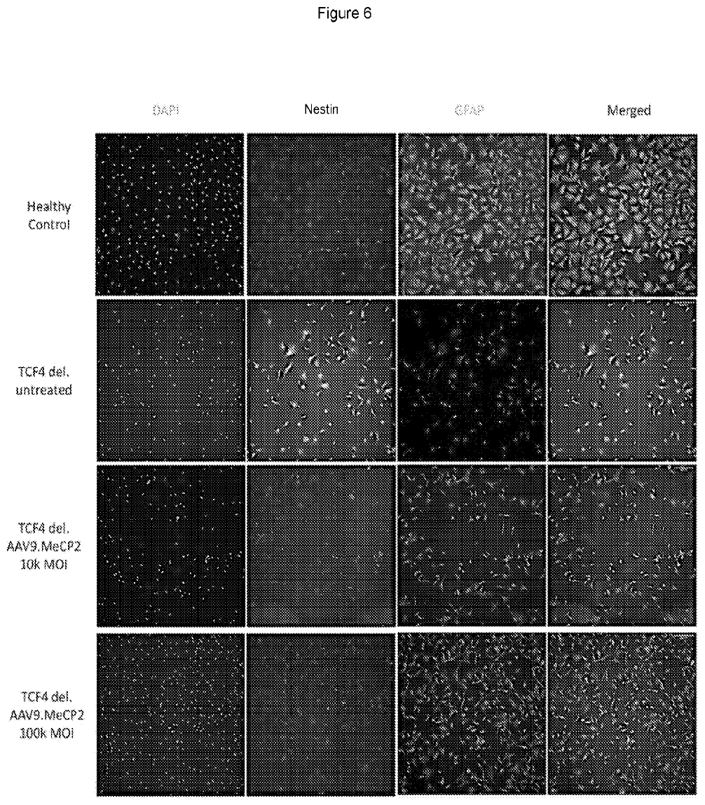

Western blot data indicating reduced MECP2 levels in the NPCs of all patient lines tested were reduced (FIG. 5). Interestingly, PTHS iAs with mutations leading to a TCF4 gene deletion also had a negative impact on iAstrocyte differentiation (FIG. 2). Furthermore, co-culture analysis on iAstrocytes derived from PTHS patients are less supportive to neurons, providing a platform to screen potential therapeutic approaches (FIGS. 4A and B). The observed reduced MECP2 levels in NPCs with TCF4 mutations suggests a restoration of MECP2 is a promising approach to treat PTHS. This is further supported by the restoration of differentiation observed when NPCs containing a TCF4 deletion mutation were transduced with MECP2.AAV9 (FIG. 6). Thus, MECP2.AAV9 gene therapy may be used to treat PTHS.

### Example 2

Constructions of the scAAV9.P546.MECP2

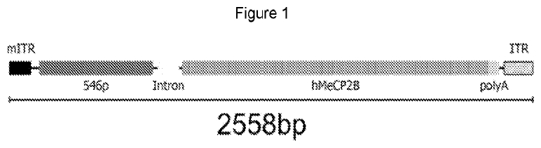

The recombinant viral genome of scAAV9.P546.MECP2 (SEQ ID NO: 5; shown in FIG. 1) includes 546 promoter (P546 promoter) driving express of the human MECP2 cDNA, and a synthetic polyadenylation signal. The gene cassette (nucleotides 151-2558 of SEQ ID NO: 5) is flanked by a mutant AAV2 inverted terminal repeat (ITR) and a wild type AAV2 ITR that enable packaging of self-complementary AAV genomes.

TABLE 4
Molecular Features of plasmid scAAV9.P546.MECP2 (SEQ ID NO: 5)

| TYPE | START | END | NAME | DESCRIPTION | SEQ ID No. |
|---|---|---|---|---|---|
| REGION | 1 | 106 | 5′ ITR | Mutant AAV2 inverted | 6 |
| | | | | terminal repeat | |
| REGION | 151 | 699 | P546 | MECP2 truncated promotor | 2 |
| REGION | 729 | 829 | SV40 | SV40 intronic sequence | 7 |
| | | | intron | | |
| GENE | 848 | 2344 | MECP2 | Human MECP2 coding | 3 |
| | | | cDNA | region | |
| REGION | 2345 | 2393 | PolyA | Synthetic PolyA | 4 |
| REGION | 2418 | 2558 | 3′ ITR | Wild-type AAV2 inverted | 8 |
| | | | | terminal repeat | |
| GENE | 3309 | 4259 | KanR | Kanamycin resistance gene | |
| REGION | 4325 | 4939 | Ori | Plasmid origin of replication | |

Self-complementary AAV9 (scAAV9) was produced by transient transfection procedures using a double-stranded AAV2-ITR-based vector, with a plasmid encoding Rep2Cap9 sequence as previously described [Gao et al., J. Virol., 78: 6381-6388 (2004)] along with an adenoviral helper plasmid pHelper (Stratagene, Santa Clara, CA) in 293 cells. Virus was produced and purified by two cesium chloride density gradient purification steps, dialyzed against PBS and formulated with 0.001% Pluronic-F68 to prevent virus aggregation and stored at 4° C. All vector preparations were titered by quantitative PCR using Taq-Man technology. Purity of vectors was assessed by 4-12% sodium dodecyl sulfate-acrylamide gel electrophoresis and silver staining (Invitrogen, Carlsbad, CA).

scAAV9 was produced by transient transfection procedures using a double-stranded AAV2-ITR-based vector, with a plasmid encoding Rep2Cap9 sequence as previously described [Gao et al., supra] along with an adenoviral helper plasmid pHelper (Stratagene, Santa Clara, CA) in 293 cells. Virus was produced in three separate batches for the experiments and purified by two cesium chloride density gradient purification steps, dialyzed against PBS and formulated with 0.001% Pluronic-F68 to prevent virus aggregation and stored at 4° C. All vector preparations were titered by quantitative PCR using Taq-Man technology. Purity of vectors was assessed by 4-12% sodium dodecyl sulfate-acrylamide gel electrophoresis and silver staining (Invitrogen, Carlsbad, CA).

The scAAV9.P546.MECP2 was tested to determine if TCF4 deletion mutation impairs iAstrocyte differentiation from Neuronal Progenitor Cells (NPCs). Healthy NPCs efficiently differentiate into induced astrocytes (iAs) as shown by reduced nestin (progenitor cell marker, green) and increased GFAP (astrocyte marker, purple) staining. TCF4 deletions (untreated) lead to a reduced differentiation efficiency as demonstrated by increased nestin and reduced GFAP staining. As shown in FIG. 2, transduction of TCF4 knockout patient NPCs in vitro with scAAV9.P546.MECP2 (10 and 100 MOI) two days prior to differentiation resulted in restored differentiation of iAs. The data demonstrates that raising expression levels of MECP2 improves iAstrocyte differentiation from NPCs.

This scAAV is also described in International Applciation Publication No. WO 2018/094251 and US Patent Application No. 20200179467, both incorporated by reference herein in their entirety. The following studies were disclosed in these application and provide data on scAAV9 expression in the wild type mice and nonhuman primates.

### Example 3

### Data in Wild-Type Mice and Non-Human Primates

Treatment of Wild Type Mice with scAAV9.P546.MECP2 Is Safe and Well Tolerated

An important concern for an MECP2 replacement therapy is to assess the impact on the cells expressing an intact copy of MECP2. scAAV9.P546.Mecp2 was designed with this consideration in mind by incorporating a fragment of the murine Mecp2 promoter to support physiological regulation of the MECP2 transgene. To test the safety of scAAV9.P546.MECP2, survival and behavior analysis was performed on cohorts of wild type mice that received P1 ICV injections of scAAV9.P546.MECP2.

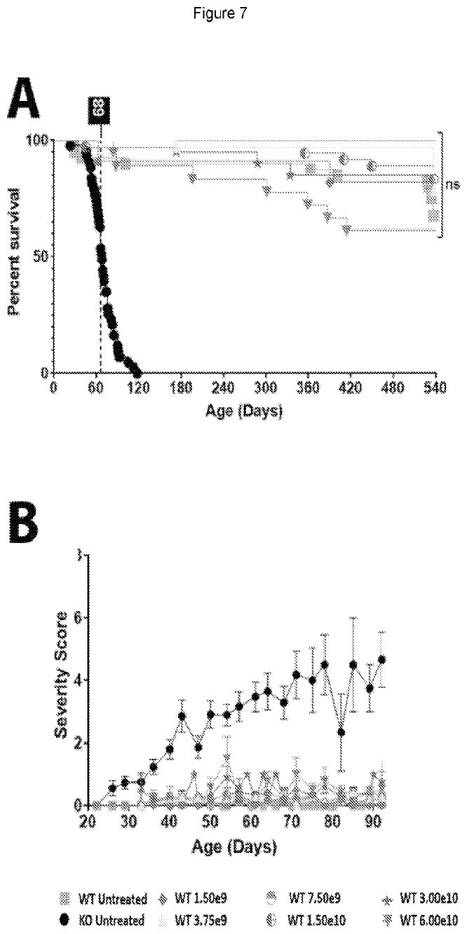

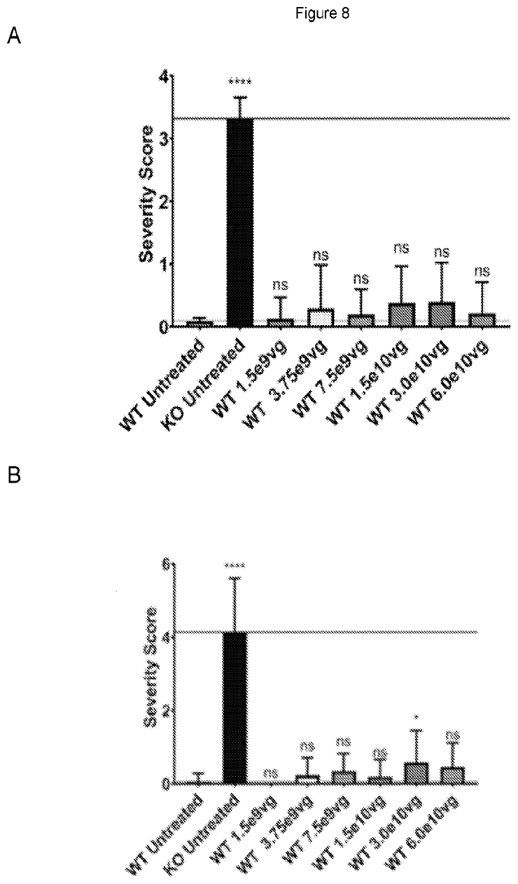

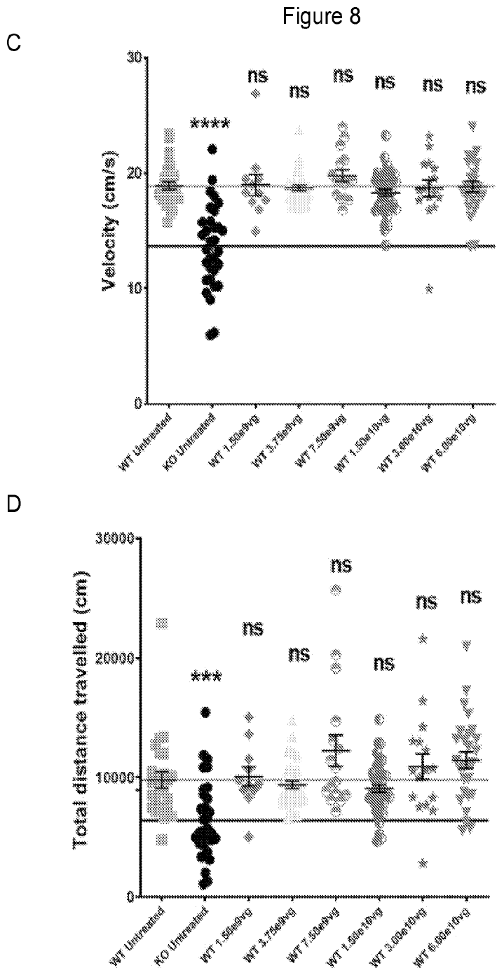

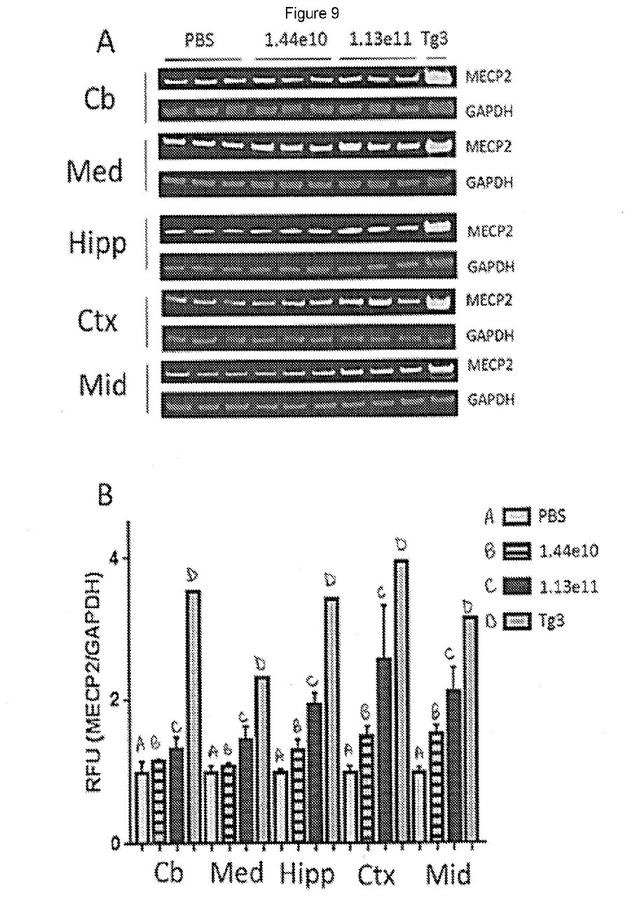

A total of 131 wild type male mice were treated with various ICV doses of AVXS- 201 and followed for survival (FIG. 7A). No deaths were recorded in the targeted therapeutic dose (1.44×10¹⁰ vg) with 21 treated animals alive through P342. No deaths were recorded in the PBS treated group and one death each was recorded in the 3.50×10⁹, 2.78×10¹⁰ and 1.13×10¹¹ vg treated groups. Behavioral scoring using the criteria from Box 1, shows that vector treated groups largely had mean phenotypic scores 1 were only noted in the two highest dosed groups (5.56×10¹⁰ and 1.13×10¹¹ vg, FIG. 7B). Open field testing at 2-3 months of age showed no statistical difference between vector and PBS treated wild type males (FIG. 8A-B). Interestingly, a significant decrease in rotarod performance was detected in the 1.13×10¹¹ vg cohort compared to control treated wild type mice at three months of age (FIG. 8C). These combined data are suggestive of a toxic effect of MECP2 overexpression at the highest AVXS-201 dose. Together these data indicate that in a “worst- case scenario” of scAAV9.P546.MECP2 treatment only transducing wild type cells, there is minimal impact on animal survival and behavior at the targeted therapeutic dose.

Physiological Levels of MECP2 are Maintained in Brains of Wild Type Mice Treated with Therapeutic Doses of scAAV9.P546.MECP2

To further investigate the levels associated with symptomatic MECP2 overexpression, wild type male mice received P1 ICV injections of PBS or scAAV9.P546.MECP2 at the therapeutic target of 1.44×10¹⁰ vg or the highest dose tested of 1.13×10¹¹ vg. Animals were euthanized 3 weeks post injection, and brains were harvested for western blot. For comparison, tissues were blotted alongside brains from a mouse model of MECP2 overexpression called Tg3. Brains were dissected into separate regions (Cb=cerebellum, Med=medulla, Hipp=hippocampus, Ctx=cortex and Mid=midbrain; FIG. 9) and the individual regions were homogenized for blotting. Data was normalized to MECP2 levels in PBS treated wild type brains. Treatment with the target therapeutic dose (1.44×10¹⁰ vg) had MECP2 levels between 1 and 1.5× wild type tissues across all regions examined. The high dose (1.13×10¹¹ vg) ranged from 1.31-2.56× wild type levels, but did not reach the 2.31-3.93× levels of Tg3 tissues (FIG. 9B). These data, along with behavior and survival data shown earlier, give confidence that scAAV9.P546.MECP2 expresses protein at near physiological levels when administered at the targeted dose. Importantly, therapeutic dosing dose not approach the 2× protein levels associated with MECP2 duplication syndrome. This shows the safety of an MECP2 replacement approach using gene therapy.

Body Weight, Hematology and Serum Chemistry are Unremarkable in Non-Human Primates through 18 Months after Intrathecal Injection of scAAV9.P546.MECP2

To investigate the safety and tolerability of scAAV9.P546.MECP2 and the associated intrathecal injection procedure, three treated male cynomolgus macaques were followed for 18 months post injection. Dosing parameters are shown in Table 5.

TABLE 5

| | | | Body Weight | Vector | | |
|---|---|---|---|---|---|---|
| | Animal | Total Viral | at Injection | Genomes/Body | Duration | |
| | ID | Genomes (vg) | (kg) | Weight (vg/kg) | post Tx | |
| Hematology | 15C34 | 6.0 × 10¹² | 1.23 | 4.9 × 10¹² | 18 | mo |
| and Serum | 15C40 | 1.4 × 10¹³ | 1.79 | 7.8 × 10¹² | 18 | mo |
| Chemistry | 15C48 | 1.4 × 10¹³ | 1.83 | 7.7 × 10¹² | 18 | mo |
| MECP2 | 15C38 | 1.3 × 10¹³ | 1.68 | 7.7 × 10¹² | 6 | wk |
| Expression | 15C49 | 1.0 × 10¹³ | 1.30 | 7.7 × 10¹² | 6 | wk |

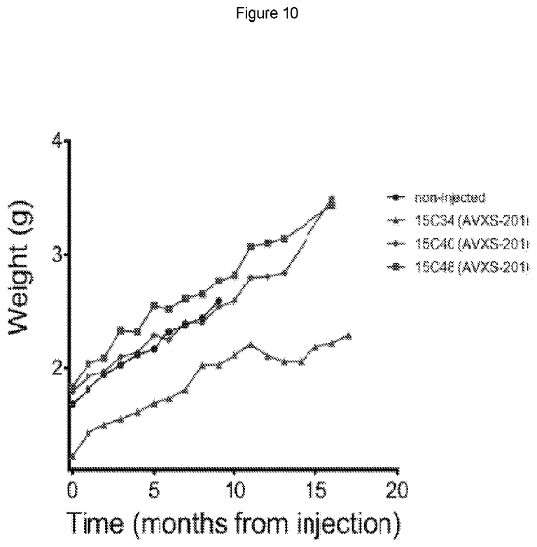

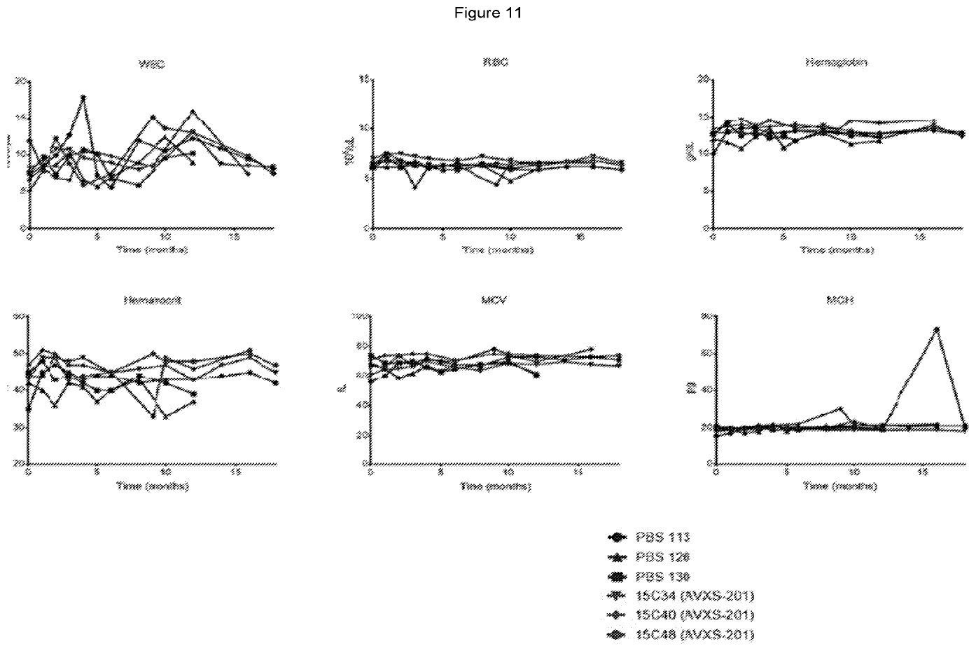

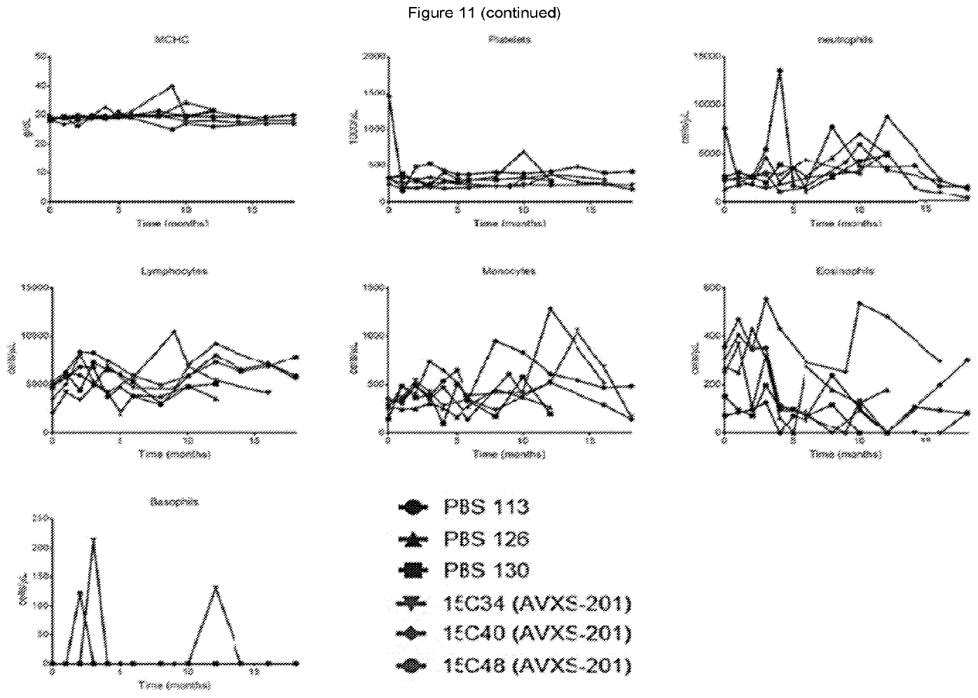

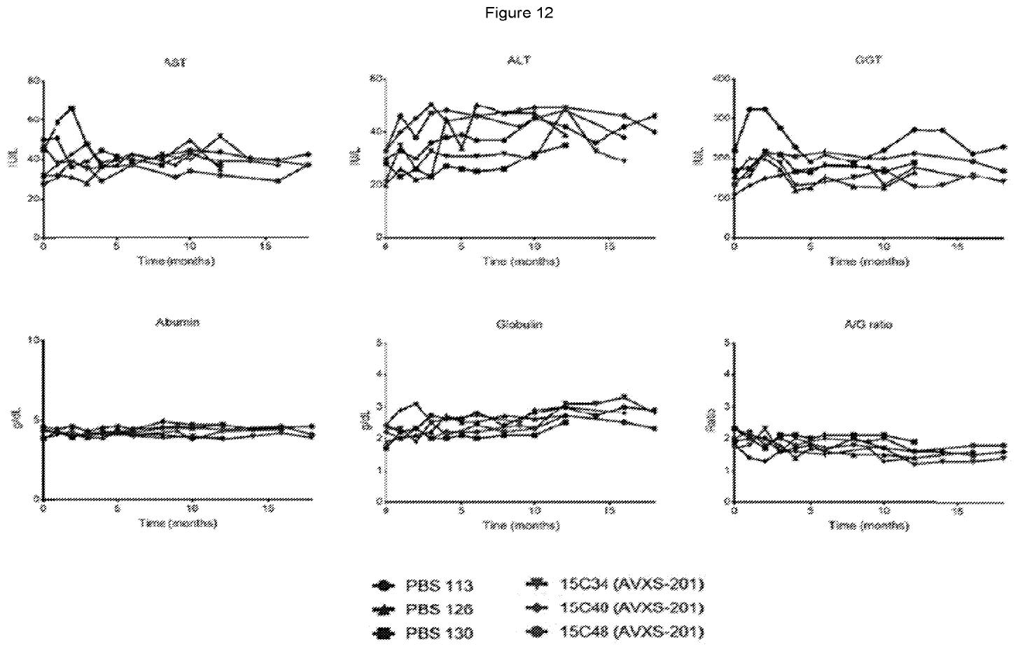

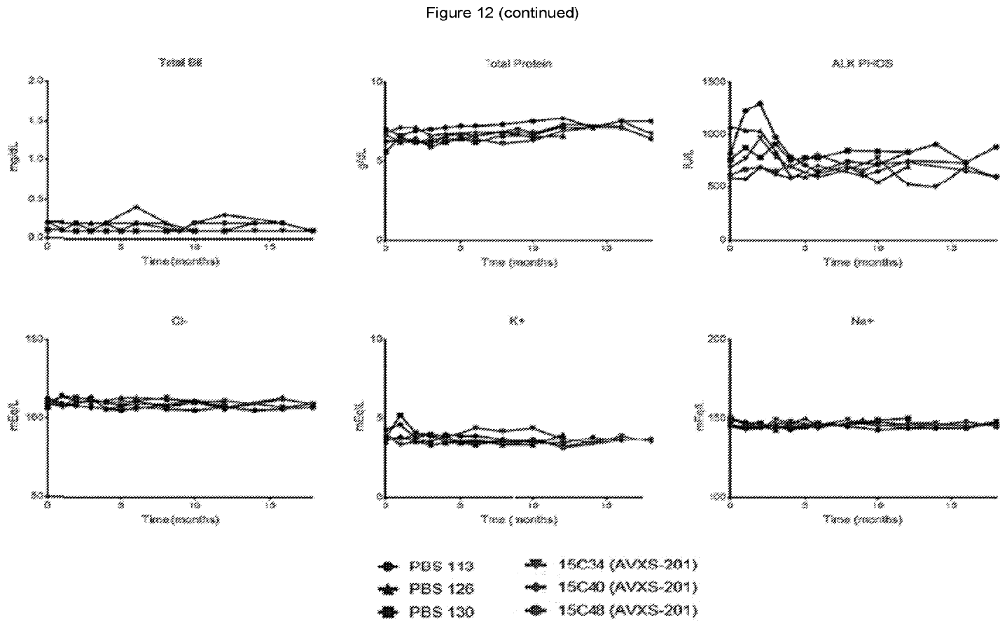

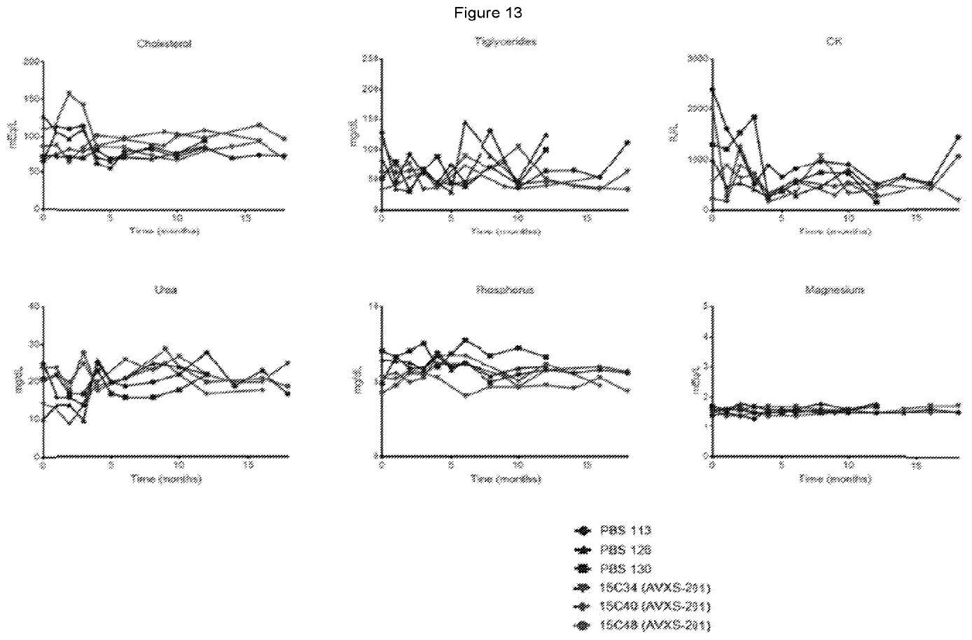

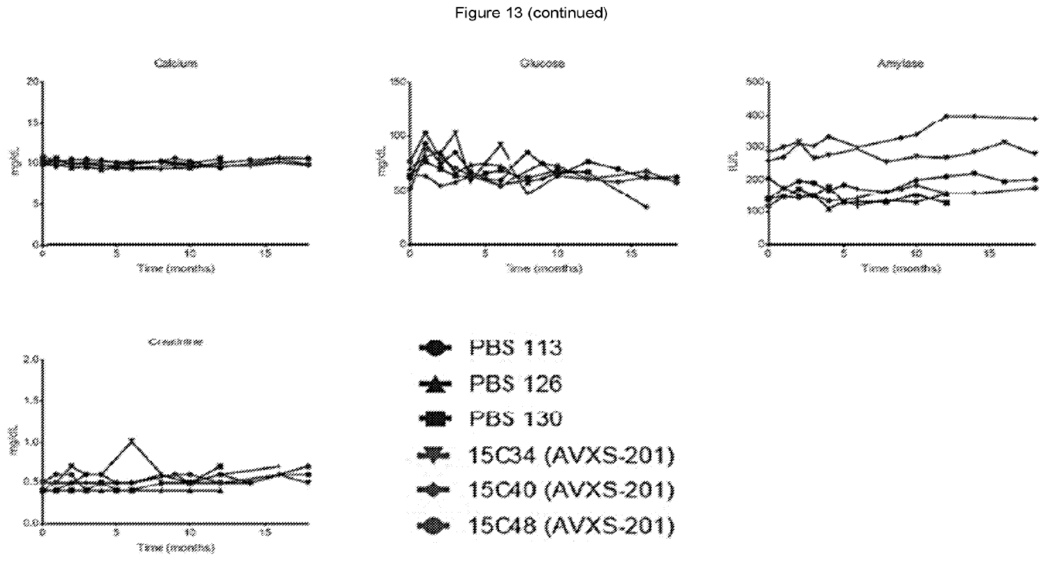

Two animals were treated at the intended therapeutic dose (˜1.44×10⁹ vg equivalent on a per kg of body weight basis), and one received a ˜2-fold lower dose (˜7.00×10⁸ vg equivalent on a per kg of body weight basis). The intrathecal injection procedure was previously described in Meyer et al., Molecular Therapy: The Journal of the American Society of Gene Therapy, 23: 477-487 (2015). Briefly, vector was mixed with contrast agent for verifying vector spread. The anesthetized subject was placed in the lateral decubitus position and the posterior midline injection site at ˜L4/5 level (below the conus of the spinal cord) was prepared. Under sterile conditions, a spinal needle with stylet was inserted and subarachnoid cannulation was confirmed with the flow of clear CSF from the needle. 0.8 ml of CSF was drained in order to decrease the pressure in the subarachnoid space and immediately after the vector solution was injected. Following injection, animals were kept in the Trendelenburg position and their body was tilted head-down for 10 minutes. Treated animals were dosed at 6 or 12 months of age, body weight, blood counts and serum chemistries were collected monthly for the first 6 months post injection, and every two months thereafter. Body weight is shown in FIG. 10, blood counts are shown in FIG. 11 and serum chemistries are shown in FIGS. 12 and 13 graphed with values from control treated animals from the same colony at the Mannheimer Foundation (Homestead, FL). Overall, body weight, cell counts and serum values from vector treated animals were consistent with control treated animals. No values substantially deviated from controls for more than 2 consecutive observations in a given animal with the exception of amylase which was higher in two vector treated animals at baseline. These data show that AVXS-201 and the intrathecal injection procedure are safe and well tolerated.

Histopathological Analysis of Tissues from Non-Human Primates Following Intrathecal Injection of scAAV9.P546.MECP2

Samples of visceral and nervous system tissues from animals 15C38, 15C49 and 15C34 (described above) were sent to GEMpath Inc. (Longmont, CO) for paraffin embedding, sectioning and hematoxylin and eosin staining. Slides were read and reports were prepared by a GEMpath Board Certified Veterinary Pathologist. The tissues sampled and examined are shown in Table 6. The pathology reports note that scAAV9.P546.MECP2 treatment did not induce lesions in any protocol-specified tissues at the 6 week or 18 month time point.

TABLE 6

| Animal ID | Tissues |
|---|---|
| 15C38 | Adrenal Gland, Brain (amygdala, striatum, |
| 15C49 | hippocampus, occipital cortex, temporal |
| | cortex, mid brain, brain stem, cerebellum), |
| | Eye and Optic Nerve, Heart, Kidney, Liver, |
| | Lung, Lymph Node (inguinal), Pancreas, |
| | Spinal Cord (sections from cervical, thoracic, |
| | lumbar and sacral regions; some sections had |
| | attached dorsal root ganglia), Small Intestine |
| | (jejunum and ileum), Skeletal Muscle (diaphragm, |
| | gastrocnemius, quadriceps femoris, triceps |
| | brachii, transverse abdominal, tibialis anterior), |
| | Spleen, Testis/Epididymis, Thymus, Urinary Bladder |
| 15C34 | Adrenal Gland, Brain (amygdala, striatum, |
| | hippocampus, hypothalamus, visual cortex, |
| | motor and somatosensory cortex, associative |
| | cortex, auditory cortex, superior and inferior |
| | colliculi, cerebellum, deep cerebellar nuclei, |
| | pons and medulla oblongata), Eye and Optic |
| | Nerve, Heart, Kidney, Liver, Lung, Lymph Node, |
| | Pancreas, Spinal Cord (sections from cervical, |
| | thoracic, lumbar and sacral regions), Small |
| | Intestine (jejunum and ileum), Skeletal Muscle |
| | (diaphragm, gastrocnemius, quadriceps femoris, |
| | triceps brachii, transverse abdominal, tibialis |
| | anterior), Spleen, Testis/Epididymis, Thymus, |
| | Urinary Bladder |

Physiological Levels of MECP2 in the Non-Human Primate Brain Following Intrathecal Injection of scAAV9.P546.MECP2

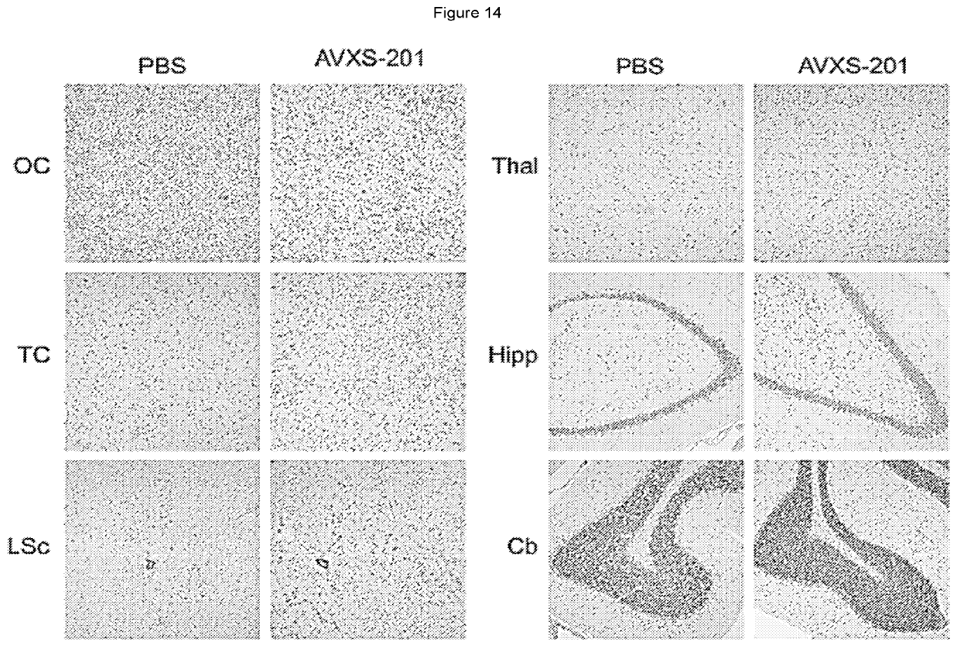

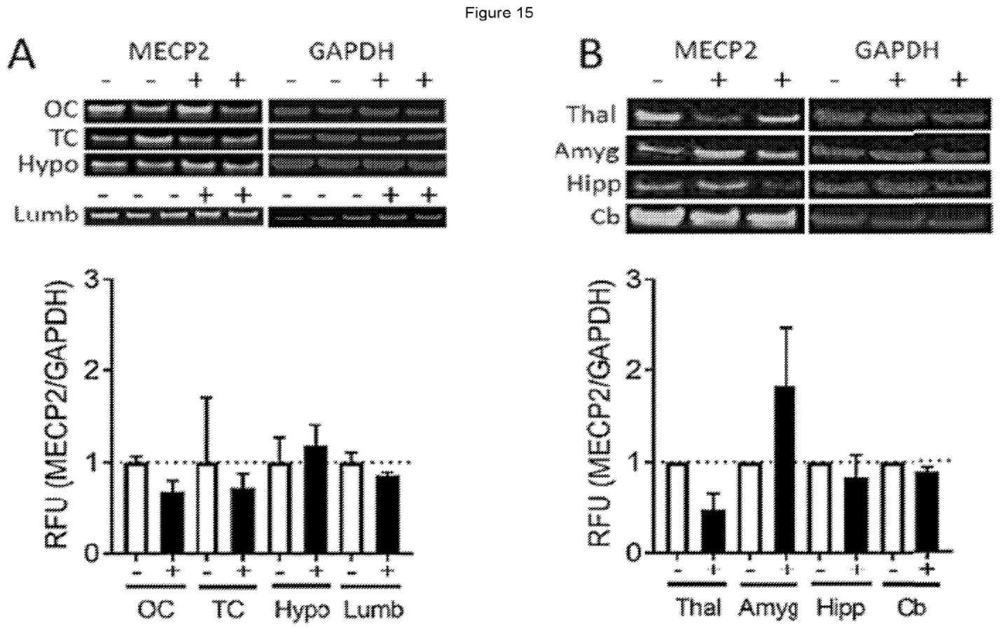

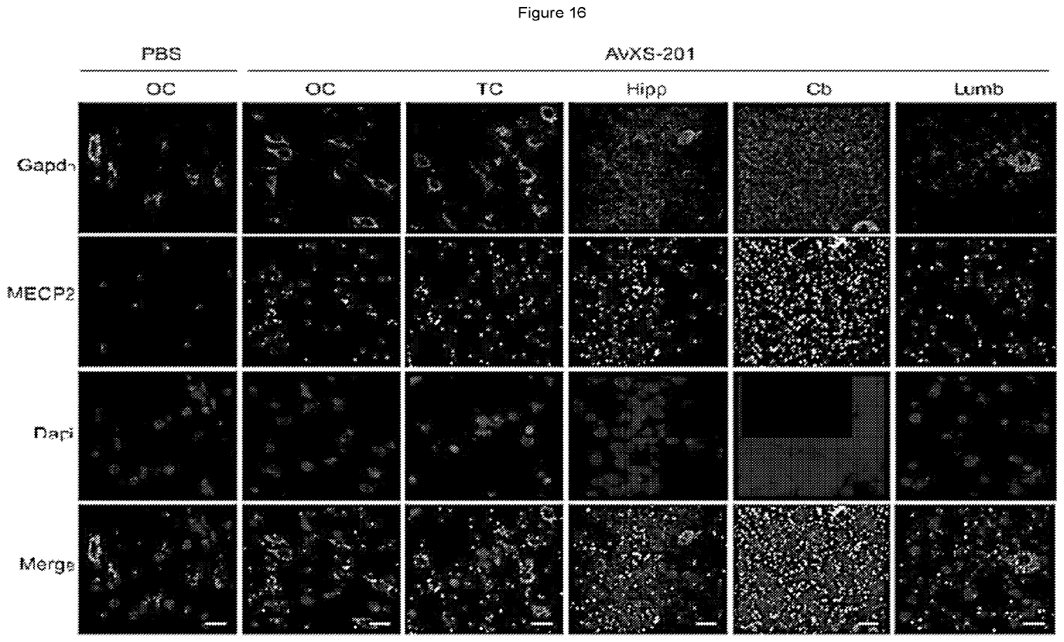

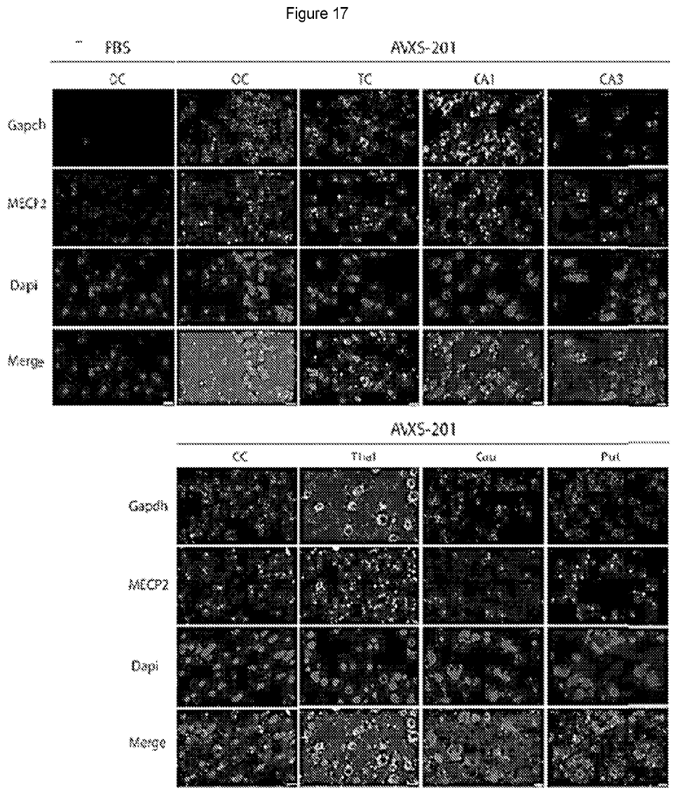

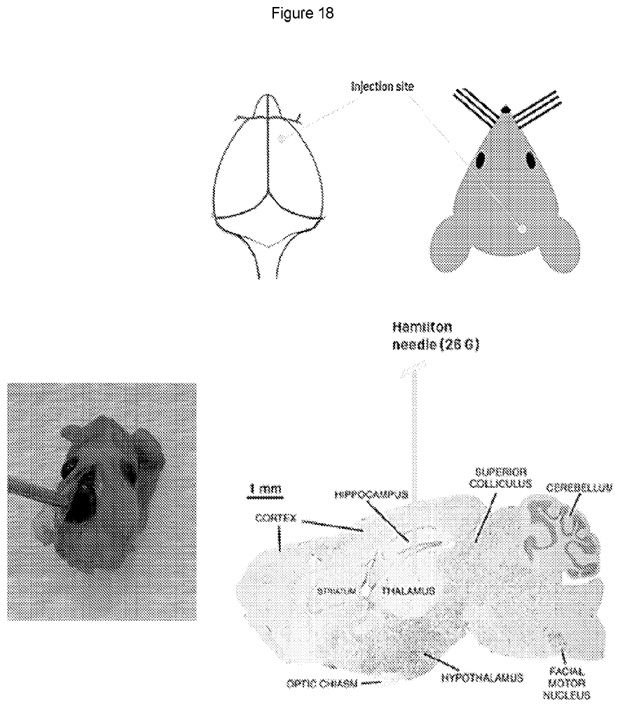

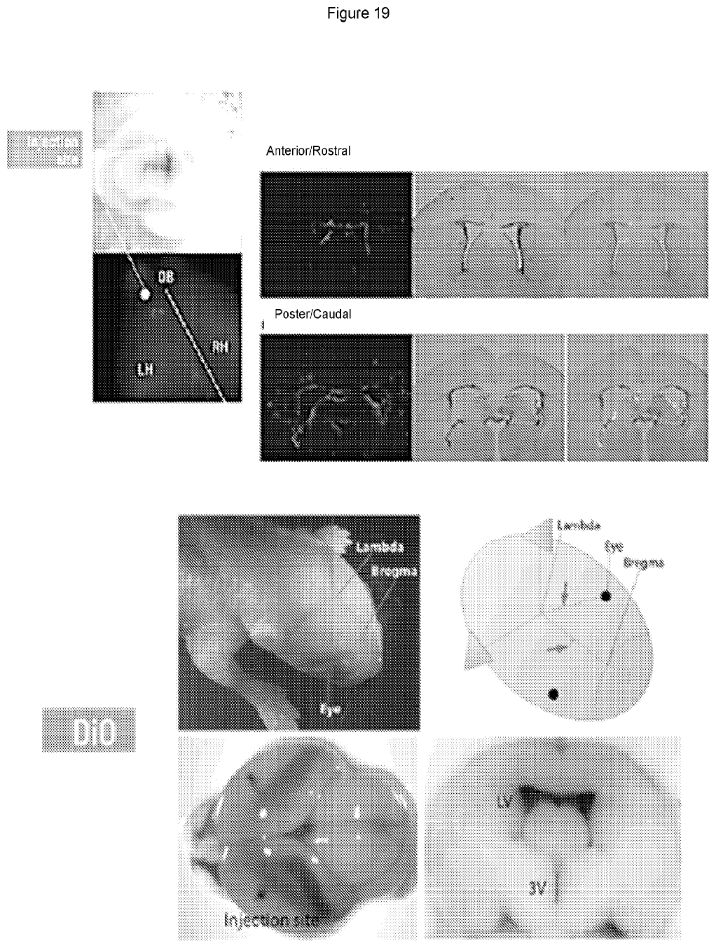

Two 12-month-old, male cynomolgus macaques received intrathecal injections of 7.7×10¹² vg/kg of AVXS-201 as described above. Animals persisted for six weeks post injection and were euthanized for analysis of MECP2 expression. Selected brain regions were analyzed for total MECP2 expression by immunohistochemistry (No obvious elevations of MECP2 were detected in cortical and subcortical regions, FIG. 14). nor proximal to the injection site (lumbar spinal cord, FIG. 14). Importantly, these data also fail to show any gross abnormalities in tissues from animals that received injection. To further examine transgene expression, brain regions were homogenized and compared against historical control tissue from animals from the same colony (FIG. 15). Samples of occipital and temporal cortices, hypothalamus, lumbar spinal cord, thalamus, amygdala, hippocampus and cerebellum were analyzed by western blot for total MECP2 expression. Across all of the regions examined no region showed a ≥2× level of MECP2 expression above controls. Elevated MECP2 was detected in the hypothalamus and amygdala which are regions proximal to 3^{rd} ventrical and lateral ventrical, respectively, but not the cerebellum. Further, the lumbar spinal cord which is proximal to the injection site did not show elevated MECP2 levels (FIG. 15). These data suggest that the combination of viral dose and expression construct are regulating MECP2 expression. Further, in situ hybridization (ISH) was performed to detect vector derived transcript and determine distribution in the brain at 6 weeks and 18 months post injection (FIGS. 16 and 17). All regions examined in the brain and spinal cord (occipital cortex, temporal cortex, hippocampus, corpus callosum, thalamus, caudate, putamen, superior colliculus, pons, medulla, cerebellum, cervical, thoracic and lumbar spinal cord) showed expression of vector derived transcript that was not present in tissues from control treated animals. These data show a specificity of the ISH probe for vector derived MECP2 transcript and show that the scAAV9.P546.MECP2 promoter construct is functional in NHP nervous system tissue. These data show that scAAV9.P546.MECP2 distributes broadly throughout the CNS when administered via lumbar puncture and expresses at physiological levels.

### Example 4

### Behavioral Analysis in TCF^{+/-} Mice

The expression of MeCP2 and the effect on behaviors in the wild type and TCF^{+/-} mice was investigated. Wild type and TCF^{+/-} mice received 1.5e10 viral genomes (vg) per animal of scAAV9.P546.GFP or scAAV9.P546.MECP2 via ICV injection within 36 hours after birth (Postnatal day 2 (P2)). The AAV was diluted in PBS to achieve a total injection volume of 5 μL per injection.

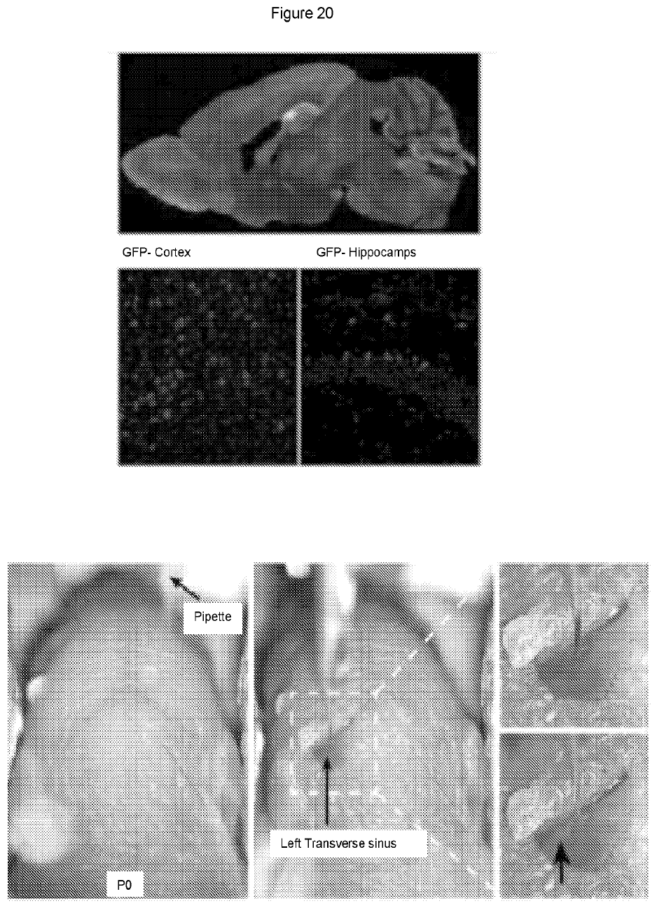

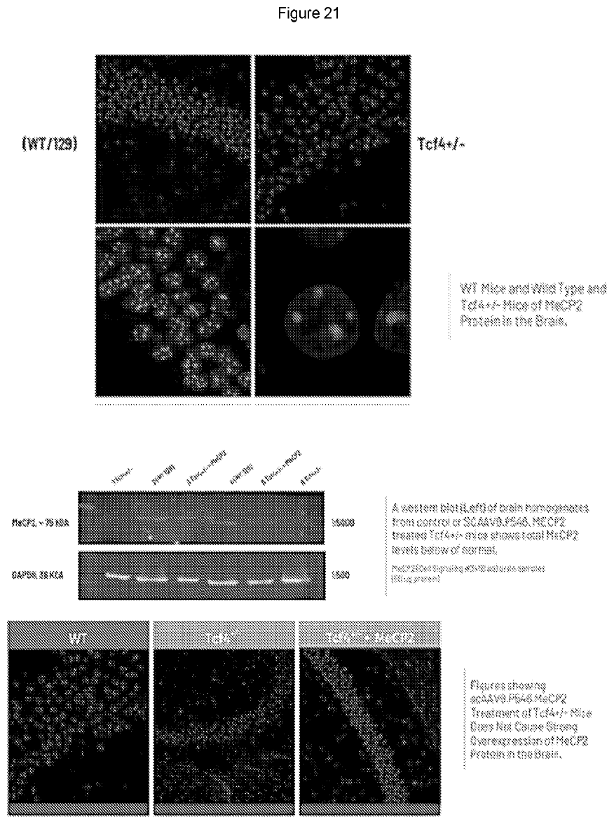

Animals were anesthetized with isoflurane in a chamber for 1 minute, Finally, the animals were decapitated and brains were removed and put in PFA 4% PBS 0.1M. The brains were cut using ice-microtone and slices were observed under epifluorescence microscope. As shown in FIG. 20, the GFP was expressed in the cortex and hippocampus of the treated mice. In addition, MeCP2 protein was expressed in the brains of the wild type and TCF^{+/-} mice after injection (see FIG. 21). This study demonstrated that injection of scAAV9.P546.MECP2 does not cause strong overexpression of MeCP2 protein in the brain.

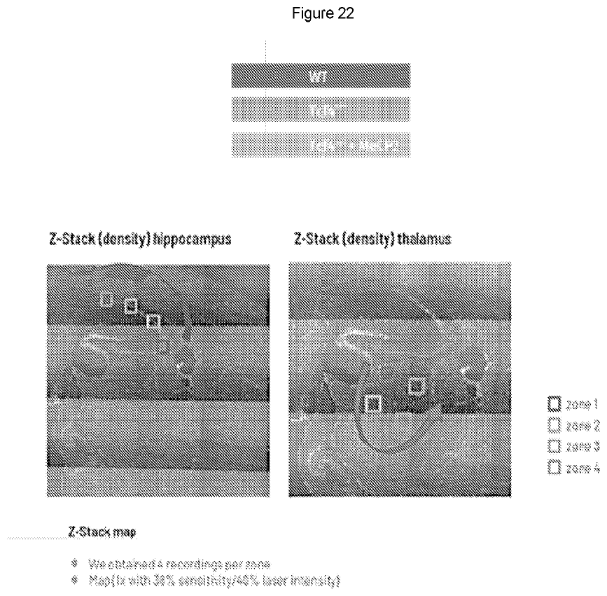

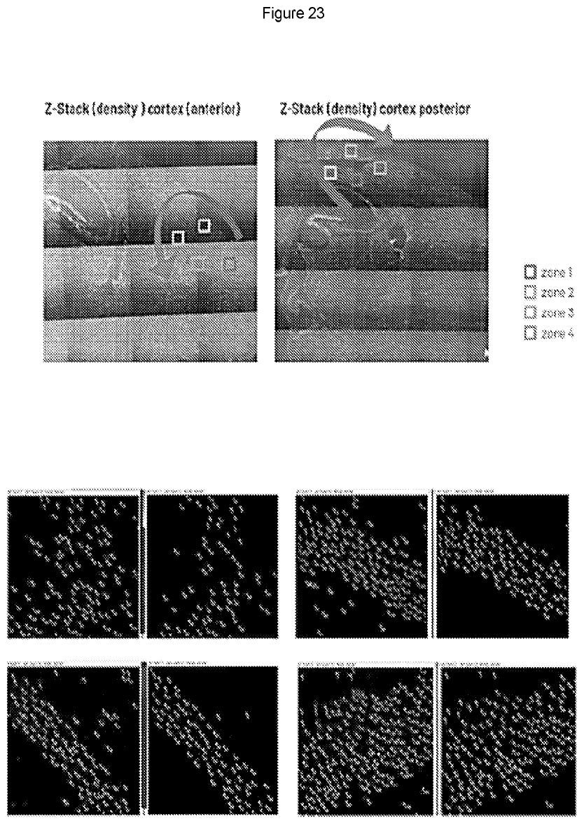

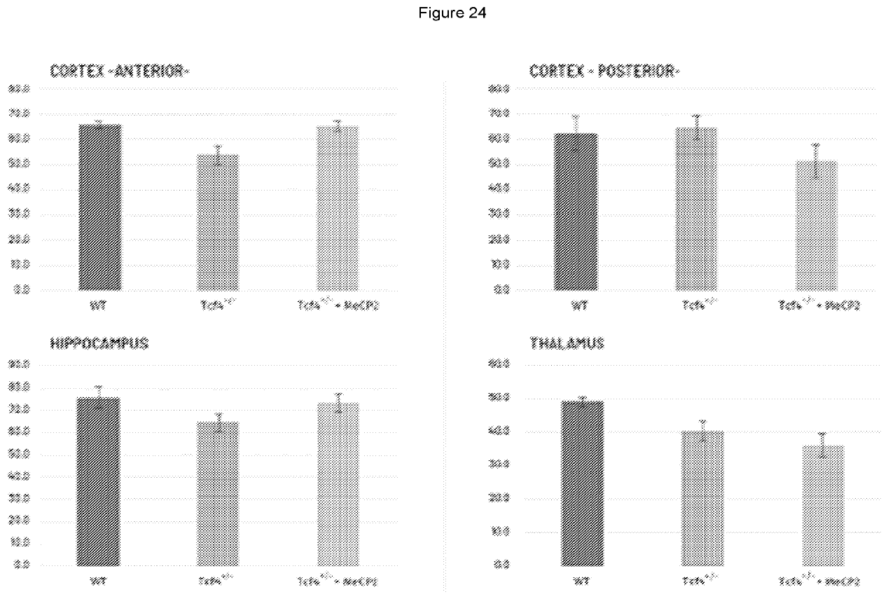

The nuclear intensity of MeCP2 protein expression in the hippocampus, cortex (anterior & posterior) and the thalamus was investigated using confocal microscopy. As shown in FIGS. 22-24, ICV injection of scAAV9.P546.MECP2 resulted in MeCP2 nuclear intensity in the cortex and hippocampus of TCF^{+/-} mice similar to that observed in wild type mice.

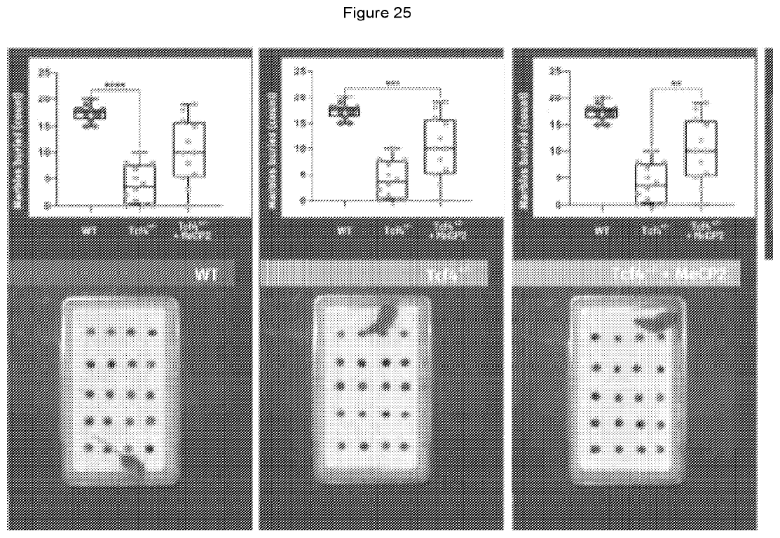

At P60, the behavior of the treated mice was analyzed. A marble burying experiment was carried out as described in Angoa-Perez et al. J. Vis. Exp. 82:50978, 2013. As shown in the FIG. 25 and the table below, ICV injection of scAAV9.P546.MECP2 improved performance in the marble burying experiment.

| | | | TCF^{+/-} |
|---|---|---|---|
| | Wild Type | TCF^{+/-} mice | mice + MeCP2 |
| Number of Values | 12 | 12 | 12 |
| Mean | 17.33 | 4.00 | 10.17 |
| Std. Deviation | 1.437 | 3.568 | 6.058 |
| Std. Error of Mean | 0.4323 | 1.030 | 1.746 |

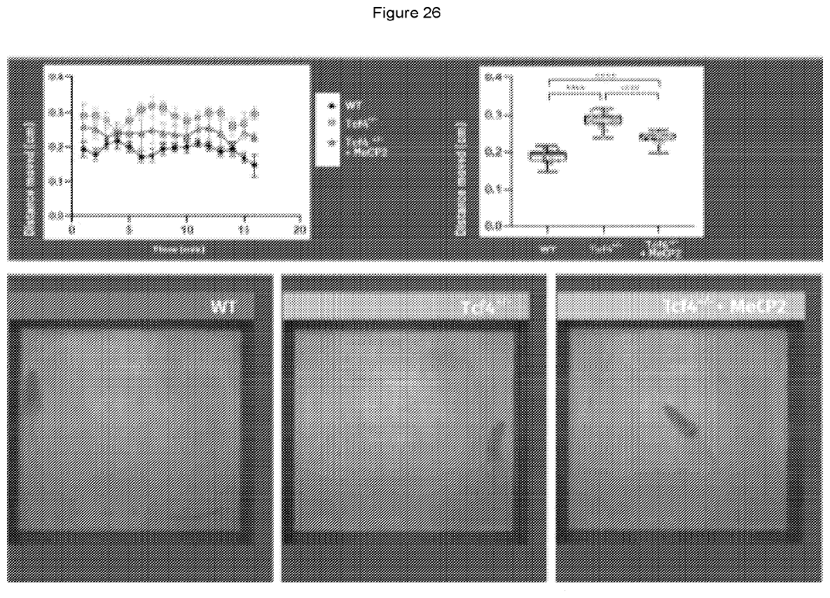

In addition, an open field test was carried out as described in Kraeuter et al. Methods Mol. Biol. 1916: 99-103, 2019. Similarly, ICV injection of scAAV9.P546.MECP2 resulted in improved performance in an open field test in TCF^{+/-} mice (FIG. 26).

| | | | TCF^{+/-} |
|---|---|---|---|
| | Wild Type | TCF^{+/-} mice | mice + MeCP2 |
| Mean | 0.1903 | 0.2857 | 0.2376 |
| Std. Deviation | 0.01836 | 0.02100 | 0.01381 |
| Std. Error of Mean | 0.004589 | 0.005249 | 0.003453 |

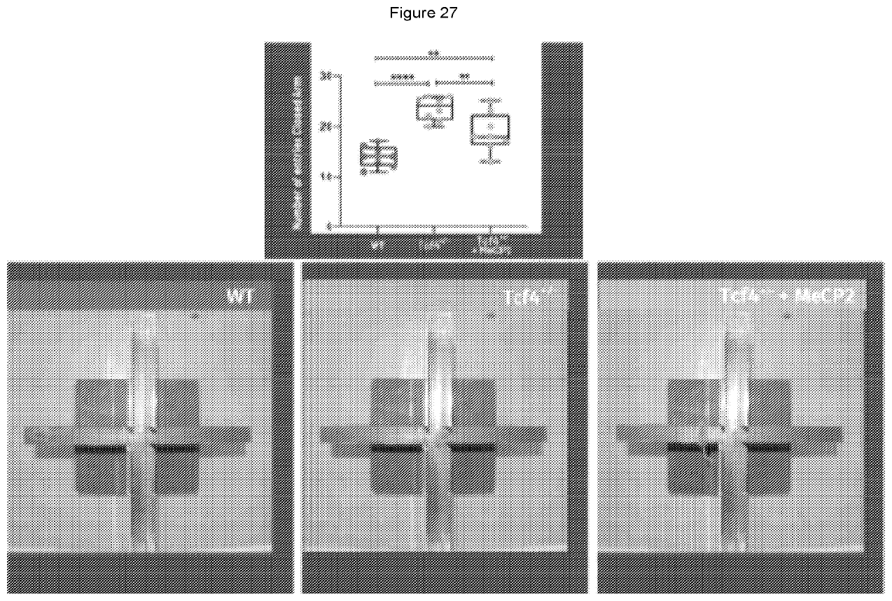

In addition, an elevated plus maze analysis was carried out as described in Komada et al. J. Vis. Exp. 22(22): 1088, 2008, and ICV injection of scAAV9.P546.MECP2 resulted in improved performance in TCF^{+/-} mice (FIG. 27).

| | | TCF^{+/-} | TCF^{+/-} |
|---|---|---|---|
| | Wild Type | mice | mice + MeCP2 |
| Mean | 14.00 | 23.50 | 18.75 |
| Std. Deviation | 2.000 | 2.330 | 3.845 |

### Example 5

### Prophetic Example in Humans

To test the potential of this construct therapeutically in humans, the

scAAV9.P546.MECP2 is delivered to the cerebrospinal fluid (CSF) of the patient. For CSF delivery, the viral vector is mixed with a contrast agent (Omnipaque or similar). For example, the compositions may comprise a non-ionic, low-osmolar contrast agent is selected from the group consisting of iobitridol, iohexol, iomeprol, iopamidol, iopentol, iopromide, ioversol, ioxilan, and combinations thereof. Immediately after lumbar CSF injection, patients will be held in a Trendelenburg position with head tilted downwards in a 15-30 degree angle for 5, 10 or 15 minutes. CSF doses will range between 1e13 viral genomes (vg) per patient—1e15 vg/patient based on age groups. New CSF delivery techniques using new injection tools developed may also be used. Intravenous delivery doses will range between 1e13 vg/kilogram (kg) body weight and 2e14 vg/kg.

While the present invention has been described in terms of various embodiments and examples, it is understood that variations and improvements will occur to those skilled in the art. Therefore, only such limitations as appear in the claims should be placed on the invention.

All documents referred to herein are incorporated by reference in their entirety.

### Sequences

P546 promoter sequence
(SEQ ID NO: 2)
GTGAACAACGCCAGGCTCCTCAACAGGCAACTTTGCTACTTCTACAGAAAATGATAATA
AAGAAATGCTGGTGAAGTCAAATGCTTATCACAATGGTGAACTACTCAGCAGGGAGGCT
CTAATAGGCGCCAAGAGCCTAGACTTCCTTAAGCGCCAGAGTCCACAAGGGCCCAGTT
AATCCTCAACATTCAAATGCTGCCCACAAAACCAGCCCCTCTGTGCCCTAGCCGCCTCT
TTTTTCCAAGTGACAGTAGAACTCCACCAATCCGCAGCTGAATGGGGTCCGCCTCTTTT
CCCTGCCTAAACAGACAGGAACTCCTGCCAATTGAGGGCGTCACCGCTAAGGCTCCGC
CCCAGCCTGGGCTCCACAACCAATGAAGGGTAATCTCGACAAAGAGCAAGGGGTGGG
GCGCGGGCGCGCAGGTGCAGCAGCACACAGGCTGGTCGGGAGGGGGGGGCGCGAC
GTCTGCCGTGCGGGGTCCCGGCATCGGTTGCGCGCGCGCTCCCTCCTCTCGGAGAGA
GGGCTGTGGTAAAACCCGTCCGGAAAAC
Coding region sequence (human MECP2 cds)
(SEQ ID NO: 3)
ATGGCCGCCGCCGCCGCCGCCGCGCCGAGCGGAGGAGGAGGAGGAGGCGAGGAGG
AGAGACTGGAAGAAAAGTCAGAAGACCAGGACCTCCAGGGCCTCAAGGACAAACCCCT
CAAGTTTAAAAAGGTGAAGAAAGATAAGAAAGAAGAGAAAGAGGGCAAGCATGAGCCC
GTGCAGCCATCAGCCCACCACTCTGCTGAGCCCGCAGAGGCAGGCAAAGCAGAGACA
TCAGAAGGGTCAGGCTCCGCCCCGGCTGTGCCGGAAGCTTCTGCCTCCCCCAAACAG
CGGCGCTCCATCATCCGTGACCGGGGACCCATGTATGATGACCCCACCCTGCCTGAAG
GCTGGACACGGAAGCTTAAGCAAAGGAAATCTGGCCGCTCTGCTGGGAAGTATGATGT
GTATTTGATCAATCCCCAGGGAAAAGCCTTTCGCTCTAAAGTGGAGTTGATTGCGTACT
TCGAAAAGGTAGGCGACACATCCCTGGACCCTAATGATTTTGACTTCACGGTAACTGGG
AGAGGGAGCCCCTCCCGGCGAGAGCAGAAACCACCTAAGAAGCCCAAATCTCCCAAA
GCTCCAGGAACTGGCAGAGGCCGGGGACGCCCCAAAGGGAGCGGCACCACGAGACC
CAAGGCGGCCACGTCAGAGGGTGTGCAGGTGAAAAGGGTCCTGGAGAAAAGTCCTGG
GAAGCTCCTTGTCAAGATGCCTTTTCAAACTTCGCCAGGGGGCAAGGCTGAGGGGGGT
GGGGCCACCACATCCACCCAGGTCATGGTGATCAAACGCCCCGGCAGGAAGCGAAAA
GCTGAGGCCGACCCTCAGGCCATTCCCAAGAAACGGGGCCGAAAGCCGGGGAGTGTG
GTGGCAGCCGCTGCCGCCGAGGCCAAAAAGAAAGCCGTGAAGGAGTCTTCTATCCGA
TCTGTGCAGGAGACCGTACTCCCCATCAAGAAGCGCAAGACCCGGGAGACGGTCAGC
ATCGAGGTCAAGGAAGTGGTGAAGCCCCTGCTGGTGTCCACCCTCGGTGAGAAGAGC
GGGAAAGGACTGAAGACCTGTAAGAGCCCTGGGCGGAAAAGCAAGGAGAGCAGCCCC
AAGGGGCGCAGCAGCAGCGCCTCCTCACCCCCCAAGAAGGAGCACCACCACCATCAC
CACCACTCAGAGTCCCCAAAGGCCCCCGTGCCACTGCTCCCACCCCTGCCCCCACCTC
CACCTGAGCCCGAGAGCTCCGAGGACCCCACCAGCCCCCCTGAGCCCCAGGACTTGA
GCAGCAGCGTCTGCAAAGAGGAGAAGATGCCCAGAGGAGGCTCACTGGAGAGCGACG
GCTGCCCCAAGGAGCCAGCTAAGACTCAGCCCGCGGTTGCCACCGCCGCCACGGCCG
CAGAAAAGTACAAACACCGAGGGGAGGGAGAGCGCAAAGACATTGTTTCATCCTCCAT
GCCAAGGCCAAACAGAGAGGAGCCTGTGGACAGCCGGACGCCCGTGACCGAGAGAGT
TAGCTGA
PolyA sequence (synthetic)
(SEQ ID NO: 4)
AATAAAAGATCTTTATTTTCATTAGATCTGTGTGTTGGTTTTTTGTGTG
scAAV9.P546.MECP2
(SEQ ID NO: 5)
ctgcgcgctcgctcgctcactgaggccgcccgggcaaagcccgggcgtcgggcgacctttggtcgcccggcctcagtgagcga
gcgagcgcgcagagagggagtggaattcacgcgtggatctgaattcaattcacgcgtggtaccacgcgtgaacaacgccagg
ctcctcaacaggcaactttgctacttctacagaaaatgataataaagaaatgctggtgaagtcaaatgcttatcacaatggtgaact
actcagcagggaggctctaataggcgccaagagcctagacttccttaagcgccagagtccacaagggcccagttaatcctcaa
cattcaaatgctgcccacaaaaccagcccctctgtgccctagccgcctcttttttccaagtgacagtagaactccaccaatccgca
gctgaatggggtccgcctcttttccctgcctaaacagacaggaactcctgccaattgagggcgtcaccgctaaggctccgcccca
gcctgggctccacaaccaatgaagggtaatctcgacaaagagcaaggggtggggcgcgggcgcgcaggtgcagcagcac
acaggctggtcgggagggcggggcgcgacgtctgccgtgcggggtcccggcatcggttgcgcgcgcgctccctcctctcggag
agagggctgtggtaaaacccgtccggaaaacgcgtcgaagggcgaattctgcagataactggtaagtttagtcttttttgtcttttatt
tcaggtcccggatccggtggtggtgcaaatcaaagaactgctcctcagtcgatgttgcctttacttctaggcctgtacggaagtgtta
ctatggccgccgccgccgccgccgcgccgagcggaggaggaggaggaggcgaggaggagagactggaagaaaagtca
gaagaccaggacctccagggcctcaaggacaaacccctcaagtttaaaaaggtgaagaaagataagaaagaagagaaag
agggcaagcatgagcccgtgcagccatcagcccaccactctgctgagcccgcagaggcaggcaaagcagagacatcaga
agggtcaggctccgccccggctgtgccggaagcttctgcctcccccaaacagcggcgctccatcatccgtgaccggggaccca
tgtatgatgaccccaccctgcctgaaggctggacacggaagcttaagcaaaggaaatctggccgctctgctgggaagtatgatg
tgtatttgatcaatccccagggaaaagcctttcgctctaaagtggagttgattgcgtacttcgaaaaggtaggcgacacatccctgg
accctaatgattttgacttcacggtaactgggagagggagcccctcccggcgagagcagaaaccacctaagaagcccaaatct
cccaaagctccaggaactggcagaggccggggacgccccaaagggagcggcaccacgagacccaaggcggccacgtca
gagggtgtgcaggtgaaaagggtcctggagaaaagtcctgggaagctccttgtcaagatgccttttcaaacttcgccagggggc
aaggctgaggggggtggggccaccacatccacccaggtcatggtgatcaaacgccccggcaggaagcgaaaagctgaggc
cgaccctcaggccattcccaagaaacggggccgaaagccggggagtgtggtggcagccgctgccgccgaggccaaaaag
aaagccgtgaaggagtcttctatccgatctgtgcaggagaccgtactccccatcaagaagcgcaagacccgggagacggtca
gcatcgaggtcaaggaagtggtgaagcccctgctggtgtccaccctcggtgagaagagcgggaaaggactgaagacctgtaa
gagccctgggcggaaaagcaaggagagcagccccaaggggcgcagcagcagcgcctcctcaccccccaagaaggagca
ccaccaccatcaccaccactcagagtccccaaaggcccccgtgccactgctcccacccctgcccccacctccacctgagcccg
agagctccgaggaccccaccagcccccctgagccccaggacttgagcagcagcgtctgcaaagaggagaagatgcccaga
ggaggctcactggagagcgacggctgccccaaggagccagctaagactcagcccgcggttgccaccgccgccacggccgc
agaaaagtacaaacaccgaggggagggagagcgcaaagacattgtttcatcctccatgccaaggccaaacagagaggagc
ctgtggacagccggacgcccgtgaccgagagagttagctgaaataaaagatctttattttcattagatctgtgtgttggttttttgtgtg
gcatgctggggagagatcgatctgaggaacccctagtgatggagttggccactccctctctgcgcgctcgctcgctcactgaggc
cgggcgaccaaaggtcgcccgacgcccgggctttgcccgggcggcctcagtgagcgagcgagcgcgcagagagggagtg
g
5′ ITR
(SEQ ID NO: 6)
Ctgcgcgctcgctcgctcactgaggccgcccgggcaaagcccgggcgtcgggcgacctttggtcgcccggcctcagtgagcg
agcgagcgcgcagagagggagtgg
SV40 Intron
(SEQ ID NO: 7)
Ggtaagtttagtcttttttgtcttttatttcaggtcccggatccggtggtggtgcaaatcaaagaactgctcctcagtcgatgttgcct
ttacttctaggc
3′ ITR
(SEQ ID NO: 8)
agg aacccctagt gatggagttg gccactccct ctctgcgcgctcgctcgctc actgaggccg ggcgaccaaa
ggtcgcccga cgcccgggct ttgcccgggcggcctcagtg agcgagcgag cgcgcagaga gggagtgg

1. A method of treating Pitt Hopkins Syndrome comprising administering a recombinant adeno-associated virus (rAAV9) encoding Methyl-CpG binding protein 2 (MECP2) to a subject in need thereof.

2. A method of increasing Methyl-CpG binding protein 2 (MECP2) levels in a subject suffering from Pitt Hopkins Syndrome comprising administering a recombinant adeno-associated virus (rAAV9) or a rAAV viral particle encoding MECP2 to the subject.

3. A method of delivering a polynucleotide sequence encoding the Methyl-CpG binding protein 2 (MECP2) to a subject suffering from PTHS comprising administering a recombinant adeno-associated virus (rAAV9) or a rAAV viral particle encoding MECP2 to the subject.

4. The method of any one of claims 1-3 wherein the rAAV comprises the nucleotide sequence of SEQ ID NO: 3.

5. The method of claim 4 wherein the rAAV further comprises the promoter sequence of SEQ ID NO: 2.

6. The method of any one of claim 4 or 5 wherein the rAAV further comprises an SV40 intron and a synthetic polyadenylation signal sequence.

7. The method of any one of claims 4-6 wherein the rAAV further comprises an inverted terminal repeat (ITR).

8. The method of claim 7 wherein the rAAV comprises a mutant ITR and a wild type ITR.

9. The method of any one of claims 1-8 wherein the rAAV comprises the nucleotide sequence of SEQ ID NO: 5.

10. The method of any one of claim 1-9, wherein the rAAV is administered using direct injection into the cerebrospinal fluid, intracerebroventricular delivery, intrathecal delivery or intravenous delivery

11. The method of any one of claims 1-10 wherein the patient has a mutation in the TCF4 gene.

12. The method of any one of claims 1-11 wherein the patient is suffering from one or more of symptoms, wherein the symptom is intellectual disability, developmental delay, breathing problems, recurrent seizures (epilepsy), and distinctive facial features, delayed or lack of speech, impaired communication skills , impaired socialization skills, hyperventilation, apnea, cyanosis, clubbing of fingers and/or toes, thin eyebrows, sunken eyes, a prominent nose with a high nasal bridge, a pronounced double curve of the upper lip (cupid's bow), a wide mouth with full lips, widely spaced teeth, thick and/or cup-shaped ears, constipation, gastrointestinal problems, microcephaly, myopia, strabismus, short stature, minor brain abnormalities, small hands and/or feet, single crease across the palm of the hands, pes planus, fleshy pads at the tips of the fingers/or toes, cryptorchidism, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems.

13. A composition for treating Pitt Hopkins Syndrome wherein the composition comprises a recombinant adeno-associated virus (rAAV9) encoding Methyl-CpG binding protein 2 (MECP2).

14. A composition for increasing Methyl-CpG binding protein 2 (MECP2) levels in a subject suffering from Pitt Hopkins Syndrome wherein the composition comprises a recombinant adeno-associated virus (rAAV9) or a rAAV viral particle encoding MECP2 to the subject.

15. A composition for delivering a polynucleotide sequence encoding the Methyl-CpG binding protein 2 (MECP2) to a subject suffering from PTHS wherein the composition comprises a recombinant adeno-associated virus (rAAV9) or a rAAV viral particle encoding MECP2 to the subject.

16. The composition of any one of claims 13-15, wherein the rAAV comprises the nucleotide sequence of SEQ ID NO: 3.

17. The composition of claim 16 wherein the rAAV further comprises the promoter sequence of SEQ ID NO: 2.

18. The composition of claim 16 or 17 wherein the rAAV further comprises an SV40 intron and a synthetic polyadenylation signal sequence.

19. The composition of any one of claims 16-18 wherein the rAAV further comprises an inverted terminal repeat (ITR).

20. The composition of claim 19 wherein the rAAV comprises a mutant ITR and a wild type ITR.

21. The composition of any one of claims 13-20 wherein the rAAV comprises the nucleotide sequence of SEQ ID NO: 5.

22. The composition of any one of claim 13-21, wherein the composition is formulated for direct injection into the cerebrospinal fluid, intracerebroventricular delivery, intrathecal delivery or intravenous delivery.

23. The composition of any one of claims 13-22 wherein the patient has a mutation in the TCF4 gene.

24. The composition of any one of claims 13-23 wherein the patient is suffering from one or more of symptoms, wherein the symptom is intellectual disability, developmental delay, breathing problems, recurrent seizures (epilepsy), and distinctive facial features, delayed or lack of speech, impaired communication skills , impaired socialization skills, hyperventilation, apnea, cyanosis, clubbing of fingers and/or toes, thin eyebrows, sunken eyes, a prominent nose with a high nasal bridge, a pronounced double curve of the upper lip (cupid's bow), a wide mouth with full lips, widely spaced teeth, thick and/or cup-shaped ears, constipation, gastrointestinal problems, microcephaly, myopia, strabismus, short stature, minor brain abnormalities, small hands and/or feet, single crease across the palm of the hands, pes planus, fleshy pads at the tips of the fingers/or toes, cryptorchidism, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems.

25. Use of a recombinant adeno-associated virus (rAAV9) encoding Methyl-CpG binding protein 2 (MECP2) for the preparation of a medicament for treating Pitt Hopkins Syndrome (PTHS) in a patient in need thereof.

26. Use of a recombinant adeno-associated virus (rAAV9) encoding Methyl-CpG binding protein 2 (MECP2) for the preparation of a medicament for increasing Methyl-CpG binding protein 2 (MECP2) levels in a subject suffering from Pitt Hopkins Syndrome

27. Use of a recombinant adeno-associated virus (rAAV9) encoding Methyl-CpG binding protein 2 (MECP2) for the preparation of a medicament for delivering a polynucleotide sequence encoding the Methyl-CpG binding protein 2 (MECP2) to a subject suffering from PTHS.

28. The use of any one of claims 25-27 wherein the rAAV comprises the nucleotide sequence of SEQ ID NO: 3.

29. The use of claim 28 wherein the rAAV further comprises the promoter sequence of SEQ ID NO: 2.

30. The use of any one of claim 28 or 29 wherein the rAAV further comprises an SV40 intron and a synthetic polyadenylation signal sequence.

31. The use of any one of claims 28-30 wherein the rAAV further comprises an inverted terminal repeat (ITR).

32. The use of claim 31 wherein the rAAV comprises a mutant ITR and a wild type ITR.

33. The use of any one of claims 25-32 wherein the rAAV comprises the nucleotide sequence of SEQ ID NO: 5.

34. The use of any one of claim 25-33, wherein the medicament is formulated to be administered using direct injection into the cerebrospinal fluid, intracerebroventricular delivery, intrathecal delivery or intravenous delivery.

35. The method of any one of claims 25-34 wherein the patient has a mutation in the TCF4 gene.

36. The method of any one of claims 25-35 wherein the patient is suffering from one or more of symptoms, wherein the symptom is intellectual disability, developmental delay, breathing problems, recurrent seizures (epilepsy), and distinctive facial features, delayed or lack of speech, impaired communication skills , impaired socialization skills, hyperventilation, apnea, cyanosis, clubbing of fingers and/or toes, thin eyebrows, sunken eyes, a prominent nose with a high nasal bridge, a pronounced double curve of the upper lip (cupid's bow), a wide mouth with full lips, widely spaced teeth, thick and/or cup-shaped ears, constipation, gastrointestinal problems, microcephaly, myopia, strabismus, short stature, minor brain abnormalities, small hands and/or feet, single crease across the palm of the hands, pes planus, fleshy pads at the tips of the fingers/or toes, cryptorchidism, stereotypic movements, involuntary hand movements, loss of gait, loss of muscle tone, scoliosis, sleep disturbances, coordination or balance problems, anxiety, behavioral problems, bruxism, excessive saliva and drooling, cardiac problems, arrhythmia, feeding problems or swallowing problems.
